(19) United States
(12) Patent Application Publication
(10) Pub. No.: US 2014/0056960 A1
(43) Pub. Date: Feb. 27, 2014

(54) Methods for Improving Fracture Healing and Bone Formation

(71) Applicants: Jagdeep Nanchahal, London (GB); Nicole Horwood, London (GB)

(72) Inventors: Jagdeep Nanchahal, London (GB); Nicole Horwood, London (GB)

(73) Assignee: Imperial Innovations Limited, Lomdon (GB)

(21) Appl. No.: 13/976,115

(22) PCT Filed: Dec. 29, 2011
(86) PCT No.: PCT/GB11/52594

§ 371 Date: Nov. 8, 2013

(30) Foreign Application Priority Data
Dec. 29, 2010 (GB) 1022060.6
May 20, 2011 (GB) 1108514.9

(51) Int. Cl. A61K 38/19; A61L 31/10; A61K 38/17

(52) U.S. Cl. CPC A61K 38/191; A61K 38/1709; A61L 31/10
USPC 424/423; 424/85.1; 514/16.7

(57) ABSTRACT

The invention provides a method of promoting bone formation in a patient at a site in need thereof, the method comprising the step of locally administering a pro-inflammatory compound to the site, wherein the pro-inflammatory compound is selected from one or more of TNF-α at optimal osteogenic dose of 0.5 to 50 ng/kg of patient body weight, or 0.01 to 3.5 μg, or 1 ng/ml or similar; IL-1β at optimal osteogenic dose of 0.1 ng/ml or similar; alarmins eg HMGB1, HMGN1, S100A8, S100A9, S100A8/9, S100A12, heat shock proteins, lactoferrin, cathelicidins, a-defensins, matrix components including versican, biglycan, fragments of hyaluronic acid and heparan sulphate; and TLR-2 or TLR-4 ligands. The invention also provides the above pro-inflammatory compounds for use in promoting bone formation in a patient at a site in need thereof. Kits comprising the compounds of the invention and a surgical implant are also provided.

The present invention is in the field of promotion of bone formation and fracture repair.

### Fragility Fractures

Osteoporosis is associated with a weakened bone structure (Barrios C, et al. Healing complications after internal fixation of trochanteric hip fractures. J Orthop Trauma 1993; 7: 438). This results in a dramatically increased failure rate of up to 50% following surgical fixation due to a ‘pull out’ or ‘cut-through’ phenomenon, whereby the screws used to secure the supporting plate fail to gain sufficient purchase in the osteoporotic bone (Cornell C. Internal fracture fixation in patients with osteoporosis. J Am Acad Orthop Surg 2003; 11: 109; Kim W Y et al. Failure of intertrochanteric fracture fixation with a dynamic hip screw in relation to pre-operative fracture stability and osteoporosis. Int Orthop 2001; 25: 360). Rates of recovery and mobilisation are limited by the time required for fracture healing as premature loading leads to implant failure; this failure accounts for the excessive morbidity and mortality seen in this vulnerable group of patients. Worldwide 100-200 million people are at risk of fragility fractures and the cost in 2000 was £1.7 billion in the UK alone (Harvey N, et al. Osteoporosis: impact on health and economics. Nat Rev Rheumatol 2010; 6:99). By 2025, the annual global incidence of hip fractures, which are the most severe with a mortality rate of 24% in the first year, is estimated at 4.5-6.3 million. The lifetime risk of clinically significant fragility fractures is 40%, equivalent to that for cardiovascular disease. There is currently no technique for accelerating healing of fragility fractures.

### High-Energy Fractures

Fractures in normal bone are also common and in the UK, approximately 3.6% of the population per annum sustain a fracture and the age-standardised lifetime fracture prevalence is 38.2% (Donaldson L J, et al. The epidemiology of fractures in England. J Epidemiol Community Health 2008; 62:174). Up to 30% of all fractures involve high-energy trauma and these largely affect young men of working age. The tibia is the most commonly fractured long bone and takes on average 43 weeks to heal, with a non-union rate of about 13% in the best centres (Bosse M J, et al. An analysis of outcomes of reconstruction or amputation after leg-threatening injuries. N Engl J Med 2002; 347:1924). Only 60% of patients eventually return to full employment following severe lower limb trauma, in part due to the long healing time. The only approved biological therapy for accelerating fracture healing is the use of bone morphogenetic proteins (BMPs). However clinical results are less impressive than in preclinical animal models (Garrison K R, et al. Bone morphogenetic protein (BMP) for fracture healing in adults. Cochrane Database Syst Rev 6:CD00695). Alternative techniques of expanding autologous mesenchymal stromal cells, including those transduced to over express BMPs, face significant translational hurdles.

There are also a group of closed fractures which are typically difficult to heal, such as those of the metatarsal shaft and neck and talar fractures.

### Orthopaedic Implants

Failure of prosthetic implants represents a further clinical problem with an ageing population. Between 1997 and 2004, the number of hip and knee replacements increased by 48% and 63%, respectively. Aseptic loosening is the leading cause of failure of prosthetic joints (Purdue P E, et al. The central role of wear debris in periprosthetic osteolysis. HSS J 2006; 2:102) and a clinical trial of BMPs was unsuccessful.

Therefore, there is an urgent need to develop strategies to accelerate bone formation, which is important both for fracture healing and around orthopaedic implants.

The present invention provides methods and compositions useful in accelerating bone formation and healing, particularly in circumstances such as those set out above, for example:

1. fragility fractures

2. high-energy and other difficult to heal fractures

3. simple, closed fractures

4. orthopaedic implants and other eg dental implants

A first aspect of the invention provides a method of promoting bone formation in a patient at a site in need thereof, the method comprising the step of locally administering a pro-inflammatory compound to the site, wherein the pro-inflammatory compound is selected from one or more of TNF-α at optimal osteogenic dose of 0.5 to 50 ng/kg of patient body weight, or 0.01 to 3.5 μg, or 1 ng/ml or similar; IL-1β at optimal osteogenic dose of 0.1 ng/ml or similar; alarmins eg HMGB1, HMGN1, S100A8, S100A9, S100A8/9, S100A12, heat shock proteins, lactoferrin, cathelicidins, a-defensins, matrix components including versican, biglycan, fragments of hyaluronic acid and heparan sulphate; and TLR-2 and/or TLR-4 ligands.

A second aspect of the invention provides the use of a pro-inflammatory compound in the manufacture of a medicament for promoting bone formation in a patient at a site in need thereof, wherein the medicament is for locally administering the compound to the site and wherein the pro-inflammatory compound is selected from one or more of TNF-α at optimal osteogenic dose of 0.5 to 50 ng/kg of patient body weight, or 0.01 to 3.5 μg, or 1 ng/ml or similar; IL-1β at optimal osteogenic dose of 0.1 ng/ml or similar; alarmins eg HMGB1, HMGN1, S100A8, S100A9, S100A8/9, S100A12, heat shock proteins, lactoferrin, cathelicidins, a-defensins, matrix components including versican, biglycan, fragments of hyaluronic acid and heparan sulphate; and TLR-2 and/or TLR-4 ligands.

A third aspect of the invention provides a pro-inflammatory compound for use in promoting bone formation in a patient at a site in need thereof, wherein the pro-inflammatory compound is selected from one or more of TNF-α at optimal osteogenic dose of 0.5 to 50 ng/kg of patient body weight, or 0.01 to 3.5 μg, or 1 ng/ml or similar; IL-1β at optimal osteogenic dose of 0.1 ng/ml or similar; alarmins eg HMGB1, HMGN1, S100A8, S100A9, S100A8/9, S100A12, heat shock proteins, lactoferrin, cathelicidins, a-defensins, matrix components including versican, biglycan, fragments of hyaluronic acid and heparan sulphate; and TLR-2 and/or TLR-4 ligands, wherein the compound is for locally administering to the site in the patient.

It is envisaged that the doses of 50 ng/kg or 1 ng/ml of TNFα listed in any of the first second and third aspects above are relevant for administration to a mouse or mammal of similar size. As discussed in the examples, it is envisaged that the optimal dosage in mice may be scaled up to appropriate levels for human patients either by using body weight or relative cross-sectional area of the tibia, as appropriate. The data provided herein suggests that the optimal osteogenic dose for local delivery of TNF-α to the site of a fracture is 10 pg to 1 ng in mice. This is intended to mean that a total of 10 pg to 1 ng of TNFα should be delivered to the site of injury to accelerate fracture healing. This total amount of TNF-α could be delivered in a solution of any concentration provided the total amount is within this range.

The average body weight of a mouse is 20 g. The average body weight of a human is 70 kg. Thus, the average human is 3500-fold larger than a mouse. Therefore, the optimal osteogenic dose for a human may be of the order of 0.035 to 3.5 μg TNFα delivered locally at the site of injury. Thus such amounts of TNF-α are included in the present invention.

With an average mouse of 20 g having an optimal osteogenic dose of TNF-α of 10 pg to 1 ng, the average amount of TNF-α required per kg body weight of the mouse may be calculated as 0.5 ng/kg to 50 ng/kg. Thus, using the calculated optimal osteogenic dose of 0.5 ng/kg to 50 ng/kg, the optimal osteogenic dose for an average human of 70 kg may be 35 ng to 3500 ng, or 0.035 to 3.5 μg of TNF-α delivered locally at the site of injury. It is envisaged that this calculation may be scaled to any size of human or other animal and is intended to be part of the invention.

The average diameter of an adult C57BI/6 mouse tibia at the fracture site in our model is 1 mm. The average diameter of a human tibia at the mid shaft is of the order of 30 mm. The human tibial cross-sectional area is 900-fold greater than the mouse. Therefore, the optimal osteogenic dose for a human may be of the order of 9 ng to 900 ng or approximately 0.01 to 1 μg TNF-α delivered locally at the site of injury. Thus such amounts of TNF-α are included in the present invention.

Thus, the optimal osteogenic dose for a human may be in the order of 0.01 to 3.5 μg TNF-α delivered locally at the site of injury based taking into account either body weight or relative tibial cross-sectional area.

It may be desirable to administer a combination of said pro-inflammatory compounds to the patient, for example administration of TNF-α with an alarmin, for example HMGB1 or S100A8, to upregulate the local effect. This may be especially relevant for non-or delayed union of fractures or patients with fractures presenting late for treatment.

The site in need of the promotion of bone formation may be any number of areas comprising bone that is injured, damaged, eroded, brittle, or defective in some other way such that it would benefit from the promotion of bone growth at that site. The promotion of bone growth is envisaged to lead to the acceleration of bone growth at that site in comparison with the rate of bone growth seen in patients who are not subject to the present invention.

Thus, the site may be a site of injury. Alternatively, the site may be a site of surgical intervention.

By “site of an injury” we include the meaning that the site may be the site of an injury, such as the fracture of a bone. By “site of surgical intervention” we include the meaning that the site may be a site of a surgical intervention, such as the insertion of an implant into a bone. The site may also be a combination of both a site of injury and a site of surgical invention. In other words, when the site is one of both an injury and a surgical intervention, this may be, for example, the placing of an implant at the site of a fracture. Alternative embodiments of such sites that fall within the intended scope of the present invention will be immediately apparent to a person of skill in the art.

The site may be a site requiring bone fusion or comprising damaged bone, eroded bone or bone defects. Such embodiments may also be found in combination with each other or with a site of injury or surgical intervention.

It is envisaged that a site where there is damaged and/or eroded bone may be more prone to injury, such as fragility fractures experienced by sufferers of conditions such as osteoporosis. Further, in patients where a site requires bone fusion, one may expect that that site may also be a site of injury, which injury may have led to the requirement for the fusion of a bone. For example, a spinal injury may call for the fusion of two vertebrae to stabilise the spine. Alternatively, it may be a site of other pathology, for example due to degeneration between vertebrae resulting in the site being treated by surgical fusion of the vertebrae.

By “site comprising bone defects” we include the meaning that the bone at that site has a defective composition or structure in comparison to healthy bone. Such defects may be congenital or they may be acquired through injury or disease or other cases as would be well known to a person of skill in the art. That a site has “bone defects” or damaged bone may be assessed, for example, radiologically (e.g. by X-ray or by CT scan), as would be appreciated by a skilled person.

The present invention may be useful in repairing damaged and/or eroded bone. By “damaged and/or eroded bone” we include the meaning that the bone has accumulated damage though environmental factors or genetic factors that have left the bone in a state of fragility and where the bone is weak and prone to fracture. Thus, the present invention may be useful in repairing bone after osteomyelitis (infection of the bone) has damaged the bone. It is also envisaged that the present invention will be useful in repairing bone damage after irradiation or chemotherapy, in patients with bone metastases of tumours or multiple myeloma. Congenital and other defects of bone may also be repaired using the methods and uses of the present invention.

It is envisaged that in any aspect of the present invention, the promotion of bone formation will aid in healing the site. As indicated above, the site may be a site of injury and/or surgical intervention. It is expected that the site of injury or surgical intervention will comprise damaged or broken bone. The promotion of bone formation is considered therefore to aid in healing fractures or fissures in the bone and in improving the strength, flexibility and/or quality of the bone and in fusing bone and/or repairing bone, as appropriate. The present invention may be particularly useful in situations where rapid healing is a high priority.

Thus, in a preferred embodiment of the present invention the site may be a site of injury and the injury may be a fracture of a bone. The present invention is considered to be particularly useful in repairing bone that has been severely fractured in a high-energy impact with periosteal stripping, such as illustrated in FIG. 1 and comminution resulting in multiple fragments.

The present invention is also considered to be useful in repairing less severely damaged bone, thus allowing the tissue layers or bone that were present at the site before the injury or surgical intervention occurred to be replenished. Such an embodiment is considered to be particularly useful after the insertion of an implant into the bone, where new bone formation is considered to enable the implant to adhere more securely than it would in the absence of the effects of the present invention. Examples include fixation of screws and implants for joint replacement.

The present invention may allow modulation of bone healing to accelerate as well as improve the quality of healing. This would allow for faster union and improved consolidation of the fracture or implant fixation. In the clinical scenario, there is a race between fracture union/consolidation and implant failure, especially in compromised bone as exemplified by fragility fractures. The invention is considered to promote union and consolidation, thereby reducing complications at the fracture or implant site and allow more rapid mobilisation of the patient.

In an embodiment of any aspect of the invention the surgical intervention may be an osteotomy. By “osteotomy” we include any surgical procedure where bone is purposefully cut to shorten, lengthen or otherwise change its alignment. The present invention is envisaged to provide a means by which the healing process, after such a procedure, may be accelerated.

In certain surgical procedures, bone grafts are utilised to accelerate growth and healing of bone. With the application of the present invention in addition to a bone graft, it is envisaged that the bone healing process may be accelerated further. An example of when a bone graft may be used includes instances when the fusion of bone is required. It is considered that the present invention will not only aid in the acceleration of the healing of a site of grafted bone but also in healing the site where the donor bone has been excised. Thus, in an embodiment the surgical intervention may be the removal of bone from a donor site for a bone graft. In a further embodiment the site of surgical intervention may be the site of a bone graft itself. It is considered that the promotion of bone formation will aid in repairing bone at the site and/or accelerating bone formation at the site.

In an embodiment of any aspect of the present invention it is considered that the promotion of bone formation at the site may aid in repairing bone at the site and/or accelerating bone formation at the site and/or increasing cortical bone volume and/or cortical bone mineral content and/or bone mineral density at the site and/or increasing mineralised volume of the healing bone and/or the mineralised bone volume fraction and/or tissue mineral density at the site and/or accelerating remodelling of the callus at the site, for example a fracture site, and/or accelerating remodelling of any newly formed bone at the site. Thus, the invention may increase the bone mineral density and/or bone volume and/or mature bone content at the site. It is further considered that the present invention may lead to an improvement in the stiffness of the bone at a fracture site. Thus, the present invention may aid in improving the strength of the newly formed bone and may reduce the likelihood of further fractures or other pathologies at that site.

In an alternative embodiment, the surgical intervention may be a procedure for inserting an implant into, around and/or adjacent to a bone. Alternatively, the surgical intervention may be for fixing an implant to a bone. Such implants may be selected from, but not limited to, the group comprising a joint replacement, a dental implant, a pin, a plate, a screw, an intradmedullary or intraosseous device. Exemplary joint replacements include hip replacements, knee replacements, shoulder replacements and elbow replacements. Dental implants may include implants into the mandible or maxilla to support crowns or other prosthetic dental structures or other implants as would be appreciated by a person of skill in the art. Further joints that may receive implants include the ankles, wrists, digits and spine. Pins and plates may be inserted to strengthen a bone or joint after an injury, such as a fracture. Promoting fixation may also be useful in osteointegrated implants, e.g. teeth, digits, facial prosthesis and hearing devices.

Current literature would suggest that the majority of implant failures in joint replacements occur as a result of loosening. Loosening can be due to infection or aseptic. In both instances, excessive inflammation is universally acknowledged to be involved in the underlying mechanism (See Dempsey et al (2007) Arthritis Research & Therapy 9: R46; Li et al (2009) BMC Musculoskeletal Disorders 10: 57; Cheng & Zhang (2008) Medical Hypotheses 71: 727-29; and Purdue et al (2006) HSS J 2: 102-13).

Intradmedullary implants are currently either inserted with cement or they are inserted uncemented/cementless. The latter have characteristics which encourage bone formation into the intraosseous component by a variety of techniques, including surface coatings such as hydroxyapatite or the presence of biocompatible material and surface characteristics which encourage bone formation and ingrowth. Biological fixation of cementless implants, as exemplified by acetabular cups in hip arthroplasty, requires initial implant stability and physical interlocking between the cup and the supporting bone to allow bone ingrowth. This is crucial for long term stability, although initial stability can be provided from a porous-coated surface or an adjunct fixation with spikes or screws, there are potential problems with these methods:

1 Coatings can cause problems including de-bonding and bead shedding

2 Spikes or screws are associated with alterations in load distribution and local bone damage during implant insertion.

Rapid ingrowth onto the acetabulum cup by a biological method of enhanced bone formation, as in the present invention, would reduce the need for adjunct fixation, expensive implant coatings and would simplify surgical technique.

Thus, it is considered that the present invention may improve adherence of the implant to the bone. By “improve adherence” we include the meaning that the adherence of the implant to the bone would be stronger in a patient who is subject to the present invention over a patient who is not. The adherence may also be more efficient and effective adherence of the implant to the bone may be achieved at an accelerated rate (i.e. occur more rapidly) compared with adherence of an implant in the absence of the methods, uses and compounds of the present invention.

Thus in an embodiment of the preceding aspect, the adherence of the implant to the bone is strengthened in comparison with adherence of an implant to bone in the absence of the present invention. The improvement in adherence may occur through newly formed bone fusing with the implant and securing the implant into place. Alternatively, or additionally, the present invention may improve the strength of the bone that is adjacent to the implant site and thus structurally improve and strengthen the area of bone housing the implant. The improved adherence of the implant to the site is envisaged to be of particular benefit where the implant is intended to remain at the site of surgical intervention for an extended period, or permanently.

The skilled person would appreciate the standard techniques in the art that are used to assess the strength and efficiency of adherence of an implant to the bone at the implant site. For example, the adherence of the implant may be measured using radiological assessment of the site. This provides for the measurement of the peri-implant bone quality and allows the physician to quantify the success of the implant. Assessment of implant adherence and the quality of the fixture may also be assessed over the longer term by assessing the stability and longevity of the implant at the site. If the fixture of the implant loosens, then the patient will generally report pain and other symptoms. Aseptic loosening accompanied by periprosthetic osteolysis is one of the leading complications of joint replacement. Thus, the present invention may reduce complications of implant surgery and improve patient quality of life. For implants used to stabilise fractures, accelerated and improved bone healing will permit faster mobilisation of the patient and reduced incidence of implant failure.

Thus, the implant may have a reduced tendency to loosening from the site of insertion in comparison with an implant inserted in the absence of the present invention. This would be evaluated by assessing patient symptoms (including pain) and assessing the patient radiologically and/or mechanically, as would be appreciated by a skilled person.

In an embodiment of the invention, the fractured bone has a disrupted or damaged periosteum and/or endosteum. By “disrupted or damaged periosteum and/or endosteum” we include the meaning that the periosteal and/or endosteal membranes that surround the bone have been broken, damaged or even completely removed such that they are no longer attached to the bone or no longer cover or envelop the bone. A common cause of a fracture resulting in a disrupted or severely traumatised periosteum and/or endosteum is a high-energy fracture. By “high-energy fracture” we include the meaning that the bone has been fractured in a high-energy collision, where a large force has collided with the bone, transferred a large amount of energy, and caused severe trauma. This commonly occurs during road-side accidents involving vehicles colliding with pedestrians, cyclists or motorcyclists at high speed. For this reason, such high-energy fractures commonly involve the bones of the lower leg, in particular the tibia. The present invention is envisaged to be useful in aiding in the healing of high-energy fractures. The endosteum is specifically removed during reaming of the medullary canal for insertion of implants with an intramedullary component.

The invention is also considered to be useful in aiding in the healing of less severe fractures and minor fractures. The types of fracture where new bone formation may be beneficial to the patient will be understood by a person of skill in the art. Thus, the present invention will be expected to be beneficial in non-periosteally stripped fractures as well as periosteally stripped fractures (see Gerstenfeld et a/, (2001) Cells Tissues Organs 169: 285-294).

Thus, it is considered that the present invention will also be beneficial in patients who have a fracture where the fractured bone has an intact periosteum and/or endosteum. Such fractures may include “closed” fractures where the soft tissue envelope has not been broken or disrupted by the impact that led to the injury. The present invention may be used in such circumstances to significantly improve and accelerate healing of the fractured bone through promoting the formation of new bone.

It is considered that the promotion of the formation of new bone by the methods, uses or compounds of the invention may be particularly useful in patients who have weakened or more brittle bone in comparison with what would be considered healthy bone. For example, such patients may have osteoporosis. Other examples of conditions or pathologies where patients may have weakened or more brittle bones and who may benefit from the methods and uses of the present invention include those who have compromised bone due to metabolic bone disorders, hereditary bone conditions, infection, malignant or benign tumours affecting bone, bone affected by chemotherapy, radiotherapy and/or disuse.

Thus, the patient may have compromised bone due to metabolic bone disorders hereditary bone conditions, osteoporosis, infection, malignant or benign tumours affecting bone, bone affected by chemotherapy, radiotherapy and/or disuse.

For example, patients with osteoporosis have reduced bone mineral density (BMD), the bone microarchitecture is disrupted, and the amount and variety of non-collagenous proteins in the bone is altered. This leads to bones that are more likely to experience fractures than healthy bone. The present invention may be used to accelerate healing and formation of bone in such patients whether they have undergone surgery or have suffered a fracture.

Thus, the present invention may lead to newly formed bone that has improved bone quality, quantity, density and shorter healing times in patients with fragility fractures in comparison with the compromised bone that was previously present at the site of injury or surgical intervention in such patients. Improved healing, gauged in terms of increased bone quality, strength, density and/or quantity could be assessed clinically, as well as by radiological techniques, including quantitative CT scanning and/or dual energy x-ray absorbiometry (DXA). DXA measures bone mineral density and may be used to quantify this variable. The present invention may also be beneficial in patients who have other bone weakening conditions such as rheumatoid arthritis, dental caries, osteomyelitis, tumour metastases in bone and multiple myeloma or radiotherapy or chemotherapy.

In an alternative embodiment, the present invention may be useful in promoting bone formation in situations where the fusion of two or more bones is required. For example, where a joint is weak or unstable, it may be desirable to fuse the bones at the joint to increase stability. For example, bone fusion may be required at the vertebrae. Where a vertebra is damaged, fusion of the damaged vertebrae to the adjacent vertebrae may improve stability of the spine. Thus, the present invention may be used to aid in the fusion of bone. Typically, in order to carry out a bone fusion, a graft of bone from another site on the patient's skeleton is taken and is transplanted at the site of desired bone fusion in order to promote bone fusion at the site of weakness. Such a bone graft may be taken, for example, from the pelvis. The bone graft (autogenous or otherwise) will typically be aided with the use of bone conducting and induction substances such as inorganic and organic matrices. Such bone induction substances may include bone morphogenetic proteins.

It is considered that the present invention is not only beneficial in promoting bone formation at the site where bone fusion is encouraged, and thus accelerating the process of bone fusion, but the present invention is also useful in promoting bone formation at the site from where the bone sample is taken, thus accelerating healing at the donor site as well.

Thus, the present invention will aid in bone fusion (e.g. joint fusion) and it is considered that the methods, uses and compounds of the present invention will replace or augment the addition of a bone graft (autogenous or otherwise), and bone conducting and/or induction substances.

In a further embodiment, the present invention may be used to address bone defects. Examples of situations that may lead to bone defects include comminution at a fracture site and bone loss (such as through fracture fragmentation, which can be segmental or periprosthetic).

In a further embodiment, the present invention may be used to augment and accelerate bone formation during distraction lengthening. Distraction lengthening may occur in, for example, the mandible and long bones.

In a further embodiment, the present invention may be used to accelerate bone formation in tissue engineered constructs. Attempts to engineer bone, either in the body or outside it, have met with limited success. The constructs have not found widespread clinical application due to the slow formation of bone capable of bearing load. The present invention may be used to accelerate bone formation and maturation of that bone in tissue engineered constructs.

In an embodiment of all aspects of the present invention, the compound may be administered (in the methods of the invention), or may be for administration (in the uses and compounds of the invention) directly to the site.

It is envisaged that the compound may be formulated as appropriate for the type of injury or surgical intervention in question. Appropriate formulations will be evident to a person of skill in the art and may include, but are not limited to, the group comprising a liquid for injection or otherwise, an infusion, a cream, a lozenge, a gel, a lotion or a paste. The compounds of the invention may also be for administration in biocompatible organic or inorganic matrices including, but not limited to, collagen or fibrinogen matrices. It is envisaged that such matrices may act as carriers of the compound in an appropriate formulation or may aid in the promotion of bone formation by augmenting the effects of the compound.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

The formulation may comprise a controlled release preparation which is biocompatible, liquid at low temperature, so suitable for injection, but assumes gel characteristics at body temperature. By “low temperature” we include the meaning of a temperature that is lower than typical, healthy, body temperature. By “body temperature” we include the meaning of the normal healthy body temperature of the organism in question, as would be understood by a person of skill in the art. It is expected that the relevant “body temperature” may be the temperature of the site of fracture or surgery, thus the preparation in this embodiment may assume gel characteristics when in contact with the affected area of the body. The relevant temperature would depend on the organism in question and on the site of fracture/surgery in question and would be readily discernable by a person of skill in the art. These preparations would also serve to localise the pro-inflammatory compound eg TNF-α at the site of delivery. Examples of such formulations include those based on Pluronic gels (F127) and ReGel™, as will be well known to those skilled in the art. The Pluronic F127 could be combined with cross-linked polyethylene glycol fibrinogen conjugates and the TNF-α combined with these matrices would be optimally delivered to the site of desired activity.

In human or animal therapy, the compounds of the invention can be administered alone but will generally be administered in admixture with a suitable pharmaceutical excipient diluent or carrier selected with regard to the intended route of administration and standard pharmaceutical practice.

The compounds of the invention are for administering locally at the site where bone healing or formation is required. They may be used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable formulations under sterile conditions is readily accomplished by standard pharmaceutical techniques well-known to those skilled in the art.

Formulations suitable for local administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Long-lived forms of the pro-inflammatory compounds may be used. For example, the half-life of a proinflammatory cytokines such as TNF-α may be increased by coupling to carrier proteins such as serum albumin and IgGFc.

The physician or veterinary practitioner will be able to determine the required dose in a given situation based on the teaching and Examples provided herein. For example, doses may be determined using techniques as described herein, for example in the Examples. The in vitro system used in FIG. 10 of PCT/gb2010/001340 may be used to determine relative potencies of different candidate compounds. This information can then be used to determine an initial dose for testing in a dose-escalation manner in appropriate patients, as well known to those skilled in the art.

For veterinary use, a compound of the invention is administered as a suitably acceptable formulation in accordance with normal veterinary practice and the veterinary surgeon will determine the dosing regimen and route of administration which will be most appropriate for a particular animal.

Endochondral bone formation proceeds through a series of discrete stages; tissue damage and disruption of blood supply are followed by haematoma formation within hours. Acute inflammation in response to traumatic injury likewise occurs within minutes/hours of the insult. Thus it will be important to enhance aspects of this phase of the response.

Thus, in a further embodiment, the compound of the present invention may be administered (or be for administration) to the site, for example, immediately following injury or surgery. Alternatively, the compound may be administered (or be for administration) at any time after initiation of surgery or after injury, for example between one hour and one year after surgery or injury. Alternatively, this may be more than one year after surgery or injury. It is preferred that the compound is administered less than a day after surgery or injury, for example up to 30 minutes, 1 hour, 2, 3, 4 or 5 hours after the injury or surgical intervention. Alternatively, the compound may be administered or be for administration multiple days, weeks or even months after the injury or surgical intervention. The later time points for administration (i.e. weeks and months after the initial insult) may be relevant, for example, where a patient has a fracture and it is treated either with surgery or a plaster cast or not treated at all and the fracture does not unite. The compound may then be for administration several months later when the non-union is diagnosed. Alternatively, the compound may be for administration at the time of surgical intervention or injury.

As noted above, it may be desirable to administer an alarmin eg HMGB1 or S100A8 before, with or after administration of TNF-α.

A further aspect of the present invention provides a kit of parts comprising a surgical implant in combination with a pro-inflammatory compound at an optimal osteogenic dose according to the first aspect. It is envisaged that the compound of the invention, in an appropriate formulation, may be applied topically with the implant, for example, as a paste or gel, a liquid or controlled release formulation or in conjunction with organic and/or inorganic matrices. In an embodiment, the kit of the invention may further comprise cement suitable for bonding the surgical implant to bone. The compound may be incorporated into the implant or into the bone cement or applied locally or administered subsequent to the placing of the implant, for example by injection. When incorporated into the cement the compound may be dispersed within the cement or coated around the cement, as would be appreciated by a person of skill in the art. Cements that are suitable for bonding the surgical implant to bone would be well known by a person of skill in the art. Currently available implant coatings include biocompatible metals and hydroxyapatite. These coatings encourage bone ingrowth but suffer from complications including debonding of the coating.

It is envisaged that the compounds of the present invention will be used in place of, or in combination with, presently available coatings. Such combinations may lead to synergistic effects including improved implant adherence and reduced complications. It is considered that the compounds of the invention may diffuse into the surrounding tissue and promote bone formation over time. It is considered that the invention will encourage bone ingrowth into a porous or biocompatible implant. Thus, in an embodiment of this aspect the surgical implant and/or cement may be coated with the pro-inflammatory compound. In a further embodiment the pro-inflammatory compound may be covalently bound to the surgical implant and/or cement. This may be via direct binding of the compound of the invention to the implant surface or it may be by direct binding to a coating enveloping the implant surface, such as a biocompatible polymeric material. It is envisaged that in this aspect of the invention the surgical implant may be a joint replacement, a dental implant, a plate, a screw, a pin, an intramedullary or intraosseous device or another suitable implant according to the earlier aspects of the invention.

In any embodiment of the present invention the patient may be selected from, but not limited to, the group comprising mammals, birds, amphibians, fish and reptiles. Exemplary mammals may be selected from, but not limited to, the group comprising humans, apes, monkeys, sheep, cattle, goats, swine, horses, dogs, cats, mice, rats, guinea pigs, hamsters, rabbits and gerbils. In a preferred embodiment of the present invention the patient is a human.

All documents referred to herein are incorporated herein, in their entirety, by reference.

The listing or discussion of a prior-published document in this specification should not necessarily be taken as an acknowledgment that the document is part of the state of the art or is common general knowledge.

The invention is now described in more detail by reference to the following, non-limiting, Figures and Examples.

### FIGURE LEGENDS

FIG. 1 Example of high-energy open tibial fracture, with comminution of bone, stripping of periosteum and loss of soft tissue envelope.

FIG. 2 Projected numbers of total primary total hip and knee replacements in the US. J Bone Joint Surg 2007. 89A: 780-5

FIG. 3 Numbers of primary and revision total hip and knee implants in the UK. Ann Rheum Dis 2004. 63: 825-30

FIG. 4A. shows histology of tibial fracture in a mouse 14 days post injury with and without local administration of TNF-α 50 ng/kg body weight (1 ng/ml) at fracture site in PBS. The TNF-α treated example shows complete bridging of the fracture site by hard callus (bone) and remodelling of the fracture callus.

FIG. 4B. shows percentage mineralisation of callus at the fracture 28 days post injury in animals where TNF-α was administered locally at the fracture site compared to untreated mice. Percentage callus mineralization was determined from the fracture site to 1 mm proximally (mean+/−SEM).

FIG. 4C. shows three dimensional reconstruction of CT scans of fracture sites of control (untreated) and TNF-α treated mice.

FIG. 5. Dose response of exogenous TNF-α in vivo. 20 μL of PBS with rhTNF-α at the corresponding dose was injected at the fracture site at days 0 and 1 in C57BI/6 mice. At day 28, mice were harvested and limbs scanned by microCT in a blinded fashion. Percentage callus mineralization was determined from the fracture site to 1 mm proximally (threshold density of 350 mg/cm³). * p<0.05, ** p<0.01.

FIG. 6. Addition of exogenous TNF-α accelerates osteoporotic fracture repair in vivo. 20 μL of rhTNF-α at 50 ng/ml was injected at the fracture site immediately post fracture and 1 day later. Osteoporosis was induced by ovariectomising mice 4 weeks prior to surgery. (A). At day 14, mice were harvested and limbs scanned by microCT in a blinded fashion. Percentage callus mineralization (threshold density of 350 mg/cm³) was significantly greater in the rhTNF-α group. ** p<0.0089. At day 28 post fracture the percentage mineralised callus volume in the three groups (normal, those that had undergone oophorectomy and oophrectomised animals treated with TNF-α) were similar. This indicates that TNF-α administered at the fracture site in an animal model of fragility fractures accelerates early healing but the final result is unchanged, i.e. TNF-α treatment accelerates physiological fracture healing. (B). 3D reconstructions show poor bridging of the control phosphate buffered saline (PBS control) treated group but bridging of the fracture site with callus and ossification of the cartilaginous intermediary as well as remodelling of the ossified callus in the rhTNF-α group.

FIG. 7A. Administration of recombinant murine IL-10, an anti-inflammatory cytokine, at the fracture site leads to inhibition of fracture healing as assessed by percentage mineralized callus volume 14 days post fracture in a mouse tibial fracture model. Percentage callus mineralization was determined from the fracture site to 1 mm proximally (mean+/−SEM).

FIG. 7B. However, inhibition of IL-10 by using an antibody to IL-10 receptor at various dosage regimes does not result in enhanced fracture healing. Therefore, inhibiting IL-10 alone is not of potential therapeutic benefit.

FIG. 8. Alarmins in human fracture supernatants. S100A8&9 levels correlate with osteogenic activity of the supernatants. Levels of S100A8&9 in supernatants from fractured (round dots) and atraumatically cut (triangles) human bone plotted against osteogenic activity of supernatant as assessed by alkaline phosphatase production by muscle derived stromal cells over 7 days. S100 A8/9 levels were determined by ELISA.

FIG. 9A. Time course of expression of alarmins at the fracture site in the mouse fracture (tibial osteotomy) model. S100A8 expression is induced by injury, especially fracture. The mice were culled and 20-40 mg of peri-fracture muscle was harvested at specific time-points post-operation: fracture (round dots) and soft tissue dissection alone (triangles); S100 A8 expression was assessed using real-time, reverse transcription PCR (HPRT1 as housekeeper gene). Maximal expression of S100A8 at the fracture site was at 12 hr post injury.

FIG. 9B. Time course of the levels of circulating alarmins in the serum in the mouse fracture model. The mice were culled at various time points post injury, blood collected and circulating S100A8 levels in the serum measured by ELISA. There was a biphasic release, with an early peak at 15 min and second peak at 24 hr. The latter would fit with the increased transcription at 12 hr at the fracture site shown in FIG. 9A. The peaks were higher in the animals undergoing soft tissue dissection as well as fracture (crosses) compared to those undergoing soft tissue dissection alone (dots).

FIG. 10. S100 A8 stimulates monocytes to produce TNF-α in a dose dependent manner via TLR-4, not TLR2 or RAGE. (A) PBMCs at 1*10⁵/ml were incubated in 10% fetal calf serum (FCS) with S100 A8 at indicated doses. There was a dose-dependent increase in TNF-α production. (B) PBMCs at 1*10⁵/ml were incubated in 10% FCS with S100 A8 at 0.5 μg/ml with the addition of either antibody to TLR4, TLR2 or isotype controls (not shown), or sRAGE over 14 hours. TNF-α levels were determined by ELISA. There was significant dose-dependent inhibition with anti-TLR-4 antibody but not with anti-TLR-2, anti-RAGE or sRAGE.

FIG. 11. TNF-α production by bone marrow cells from wild type (WT), TLR-4 and MyD88 knockout mice treated with murine S100A8. These data confirm that TNF-α production is largely dependent on TLR-4 receptor and absolutely dependent on MyD88, a cytoplasmic universal adaptor protein used by TLR-receptors including TLR-4 and TLR-2.

FIG. 12. S100 A9 and S100 A12 are much less potent than S100 A8 for PBMC stimulation. PBMCs at 1*10⁵/ml were incubated in 10% FCS with S100 A8, S100 A9 or S100 A12 at the indicated doses over 14 hours. TNF-α levels were determined by ELISA.

FIG. 13. The other pro-inflammatory cytokines that we had previously found to be osteogenic in vitro were IL-6 and IL-1β, and these are also produced by monocytes via TLR4 and also RAGE, but not TLR2. PBMCs at 1*10⁵/ml were incubated in 10% FCS with S100 A8 0.5 μg/ml with the addition of either antibody to TLR4, TLR2 or isotype controls, or sRAGE over 14 hours. IL-6 (A) and IL-16 (B) levels were determined by ELISA. These data show that whilst TNF-α production by PBMC in response to s100A8 is largely dependent on TLR-4 receptor (FIG. 10B and FIG. 11), for IL-1β and IL-6 production signaling via RAGE may also be important (FIG. 13).

FIG. 14. Fracture healing was impaired in TLR-2^{-/-} deficient mice but not in TLR-4^{-/-} animals. Percentage callus mineralization was determined from the fracture site to 1 mm proximally (mean+/−SEM). * p=0.03, ** p=0.0049

FIG. 15. Supernatant from PBMCs stimulated with S100A8 but not LPS is osteogenic. PBMCs at 1*10⁵/ml were stimulated with S100 A8 at indicated doses (μg/ml), or LPS at 10 ng/ml. The supernatants were harvested and filter-sterilised through a 0.2 μm filter. MDSCs were cultured in this S100 A8-stimulated supernatant over 7 d and alkaline phosphatase levels quantified. These data show that the osteogenic effect is specific to alarmins such as S100A8 and does not occur on with stimulation with LPS, a pathogen specific molecular pattern.

FIG. 16. Osteogenic activity of supernatant from human monocytes exposed to varying doses of LPS. These data show that even low doses of LPS, which should lead to production of TNF-α within the osteogenic range, do not result in enhanced alkaline phosphatase production by human muscle derived stromal cells.

FIG. 17. HMGB1 is not pro-inflammatory in isolation in vitro and requires complex formation with other molecules (Bianchi M E. HMGB1. J Leukoc Biol, 2009 86(3): 573-6; Hreggvidsdottir H S et al. The alarmin HMGB1 acts in synergy with endogenous and exogenous danger signals to promote inflammation. J Leukoc Biol 2009. 86(3): 573-6; Yanai H et al. HMGB proteins function as universal sentinels for nucleic-acid-mediated immune responses. Nature. 2009. 462(7269): 99-103). The addition of LPS, a TLR-ligand, induces a synergistic effect with HMGB1 in TNF-α production by peripheral blood mononuclear cells (PBMCs). PBMCs at 1*10⁵/ml were incubated in 10% FCS with HMGB1 or LPS at the indicated doses over 14 hours; TNF-α levels were determined by ELISA.

FIG. 18. Addition of exogenous alarmin rmHMGB1 locally at the fracture site in the murine model of endochondral fracture healing accelerates fracture in vivo. C57BI6 mice were subjected to fracture model with addition of 20 μL of rmHMGB1 at 50 ng/ml at the fracture site at days 0 and 1 compared to controls. Addition of exogenous S100A8 or S100A9 or S100A8/9 had no effect, possibly because the fracture environment may already contain optimal amounts of S100 A8 or A9 proteins. All mice were harvested at day 28 and limbs scanned by microCT in a blinded fashion. Percentage callus mineralization was determined from the fracture site to 1 mm proximally (mean+/−SEM).

FIG. 19. Schematic of early fracture healing pathway. The fracture leads to cell death, resulting in release of alarmins including S100A8, possibly from the cytoplasm of injured cells or neutrophils attracted to the site of injury, as well as nuclear alarmins such as HMGB1 or HMGN1. These interact with receptors, eg TLR-4 or RAGE, on cells of the monocyte lineage to lead to the release of osteogenic molecules including TNF-α. The latter promote chemotaxis of mesenchymal stromal cells and local osteogenic differentiation.

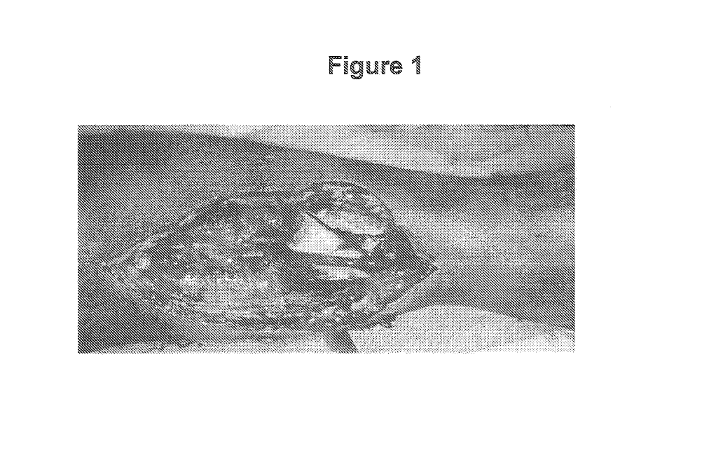

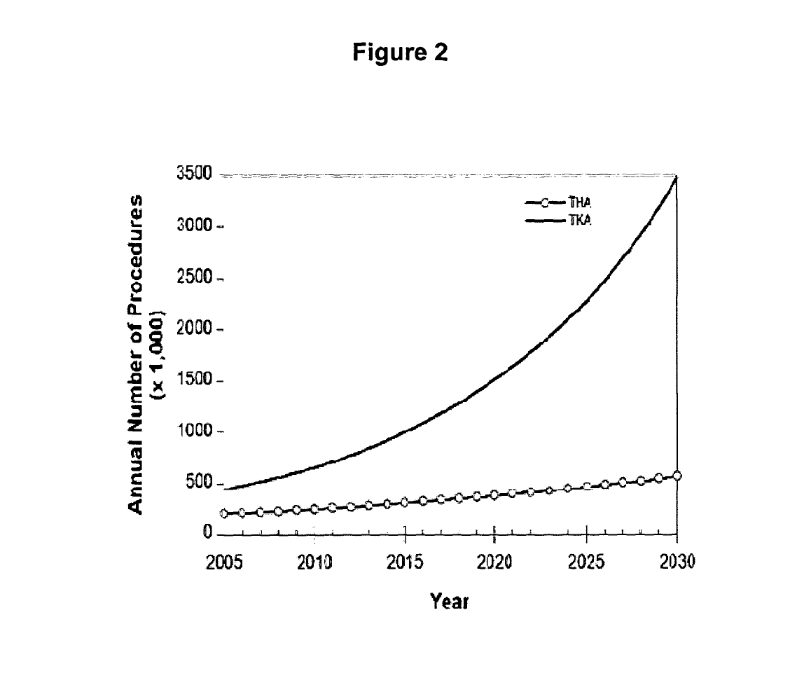

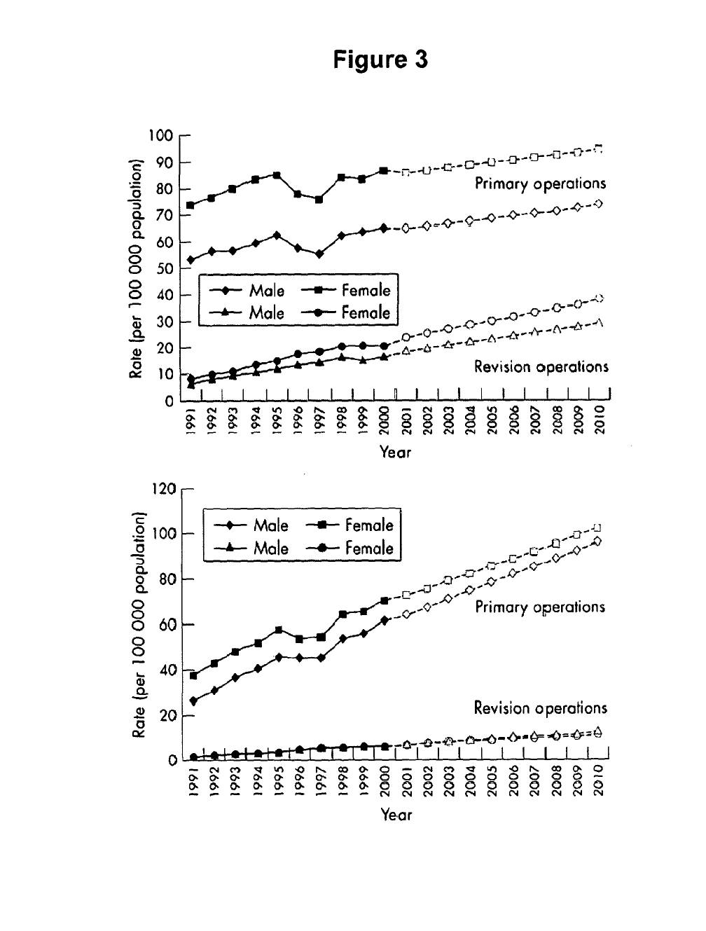

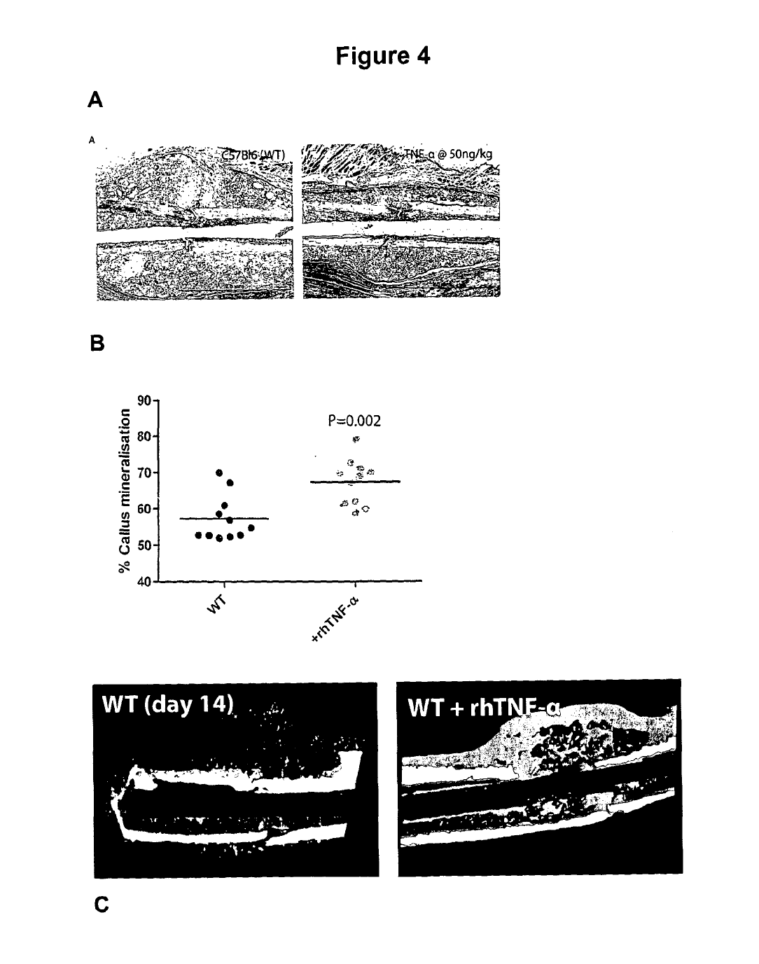

### EXAMPLE 1

### Promoting Bone Formation and Fracture Repair by Manipulating the Inflammatory Environment

TNF-α

We have demonstrated that the post fracture inflammatory milieu promotes osteogenic differentiation of muscle derived stromal cells (MDSC) and that these cells can migrate and differentiate upon stimulation with the pro-inflammatory cytokine TNF-α and to a much lesser extent by IL-6 (see PCT/GB2010/001340 and Glass et a/2011, PNAS 108(4): 1585-90). We have also shown that TNF-α, delivered locally at doses of approximately 1 ng or 50 ng/kg administered at the time of fracture and 24 hr later, is effective in promoting bone formation and accelerating fracture healing in our murine fracture model (PCT/GB2010/001340 and Glass at al 2011, PNAS 108(4): 1585-90).

TNF-α Enhances Fracture Repair within a Narrow Therapeutic Range

### IL-1β Enhances Fracture Repair

Local injection of rhTNF-α at a concentration equivalent to 50 ng/kg immediately after surgery has been shown to accelerate callus mineralization in our murine model of a periosteally-stripped (high-energy) open tibial fracture.

Although IL-1β alone does not promote bone nodule formation in vitro using human specimens, it does lead to increased alkaline phosphatase production by human muscle-derived stromal cells (PCT/GB2010/001340 and Glass at al 2011, PNAS 108(4): 1585-90). Therefore, although it has some osteogenic activity, acting alone in vitro it is unable to induce complete bone formation. TNF-α is known to lead to IL-1β production and in vivo several cytokines are known to act together to promote osteogenesis by bone marrow-derived stromal cells¹⁷.

Inflammation plays a vital role in early fracture repair^{11,12}. In murine models, TNF-α, IL-1 and IL-6 are expressed at the fracture site within 24 hours of injury^{13,14}. Biphasic expression of TNF-α and IL-1β appears to be linked to the initiation of fracture repair and later to endochondral maturation^{14,15}. In our murine model, callus mineralization at day 28 was enhanced when TNF-α, at a concentration of 50 ng/kg was injected locally following injury (day 0) and 24 hours later. Our in vitro data using human muscle-derived stromal cells demonstrated a clear concentration dependency between TNF-α and cell migration and osteogenic differentiation (PCT/GB2010/001340; Glass at al 2011, PNAS 108(4): 1585-90). Hence, we evaluated concentration-dependency of TNF-α in our murine model, using 100-fold concentration differences. In our murine model a “low” dose of 50 pg/kg is considered to result in callus mineralization at day 28 that is indistinguishable from the PBS only control. By contrast a “high” dose of 5-100 μg/kg is considered to result in significantly less callus mineralization at day 28. Our previous in vitro findings using human tissue specimens show that TNF-α accelerates bone formation within a narrow therapeutic window and is inhibitory at high concentrations.

The role of TNF-α in fracture healing has been investigated previously using in vivo models. A closed tibial fracture model using dual TNF-α receptor (p55^{-/-}/p75^{-/-}) knockout mice demonstrated impaired chrondrocyte differentiation, periosteal bridging and callus resorption¹⁶. However, daily intra-peritoneal administration of TNF-α inhibited bone formation around an intramuscular bone-inducing implant, an effect reversed on discontinuation of TNF-α⁴. The same group demonstrated, in a rib fracture model using rats, that daily intra-peritoneal administration of 400 μg/kg of TNF-α impaired healing by suppressing synthesis of the cartilaginous intermediary³. A lower dose of 40 μg/kg/day was associated with a (non significant) improvement in fracture healing over the duration of the study. Our data provide an explanation for these apparently contradictory findings.

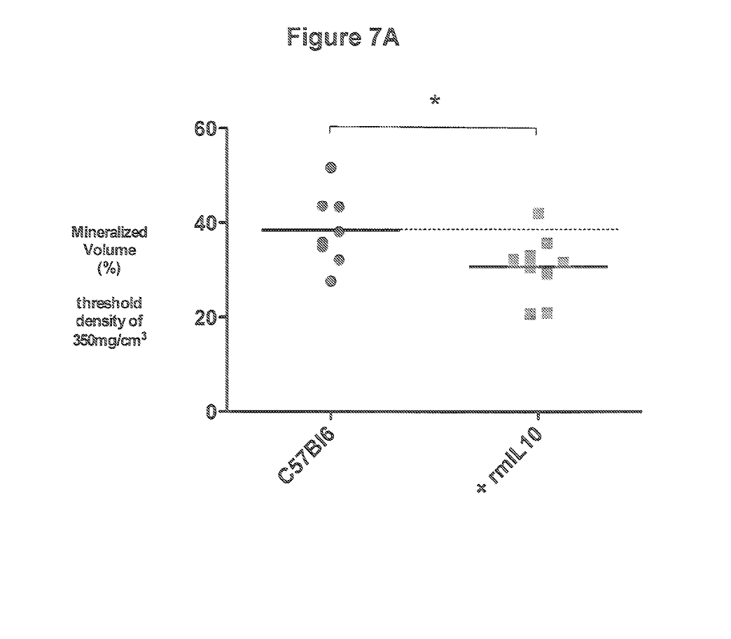

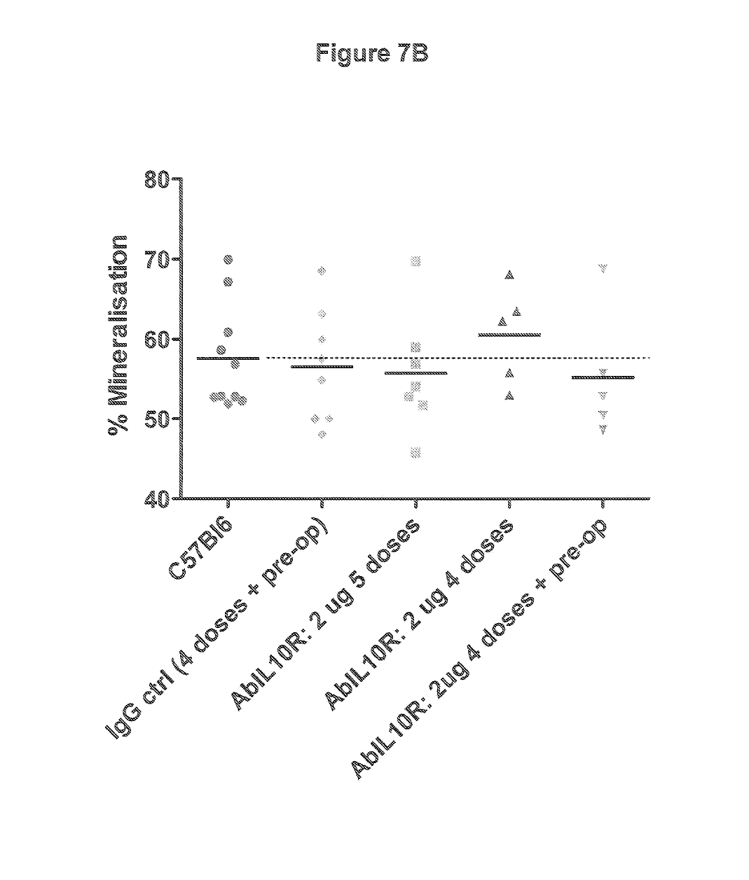

The inhibition of fracture healing seen in our murine model on addition of exogenous recombinant murine IL-10 (FIG. 7A) confirms the key role of inflammation in fracture healing and supports our data that targeted upregulation of pro-inflammatory cytokines during the early phases of healing, either soon after injury or surgical fixation, leads to accelerated callus formation and remodelling. Inhibition of the receptor for IL-10, however, did not affect fracture healing (FIG. 7B), possibly because IL-10 can act at very low concentrations, especially on dendritic cells.

Mesenchymal stromal cells do not demonstrate an osteogenic response to TNF-α alone but do in combination with other cytokines¹⁷. Moreover, we reported previously that antibody neutralization of TNF-α in supernatants derived from human fractured bone fragments impaired cell migration and osteogenic differentiation but did not wholly suppress either. These findings suggest that TNF-α acts in concert with other factors at the fracture site.

Alarmins

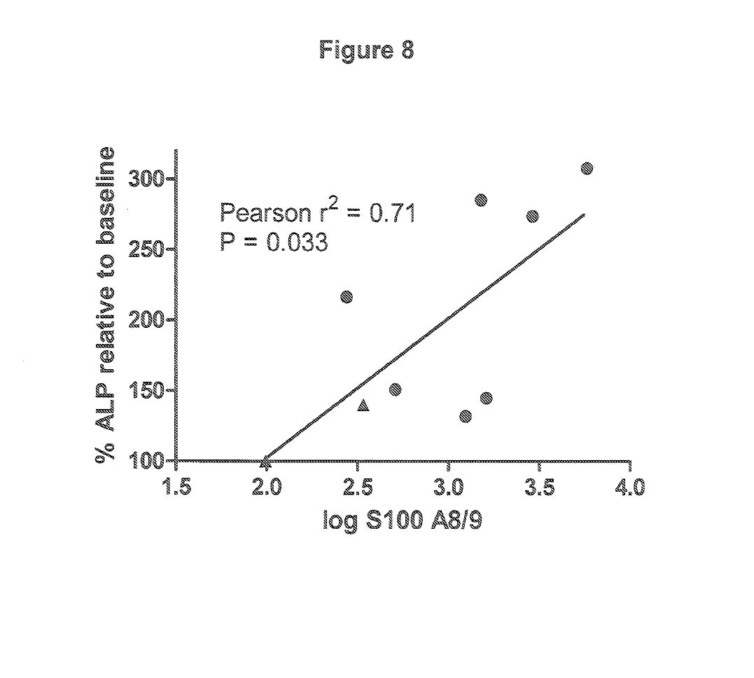

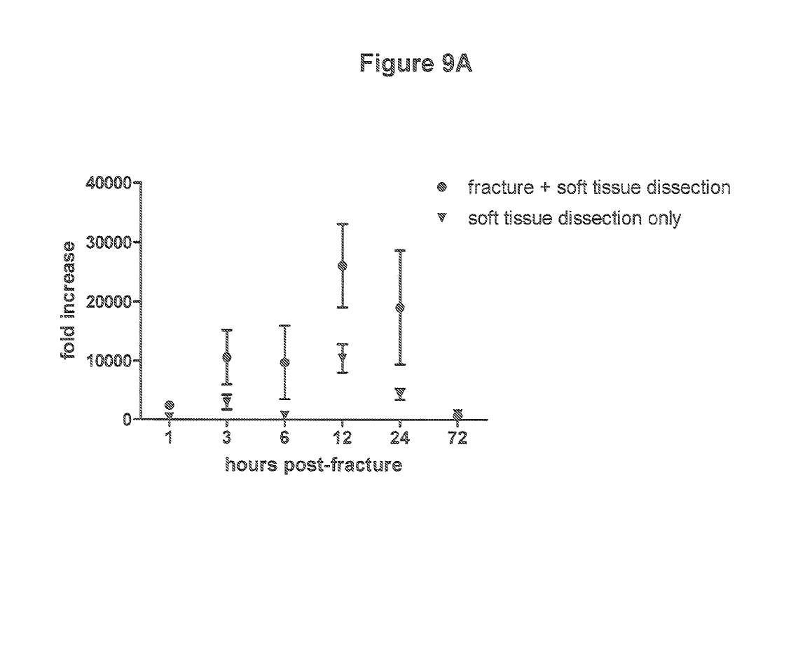

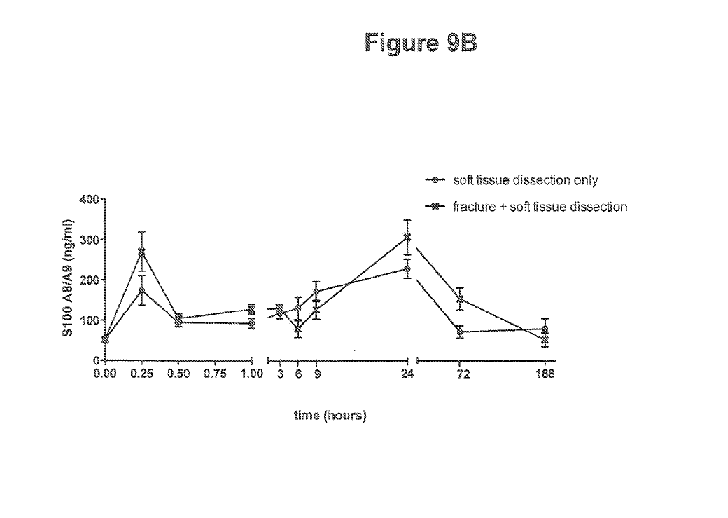

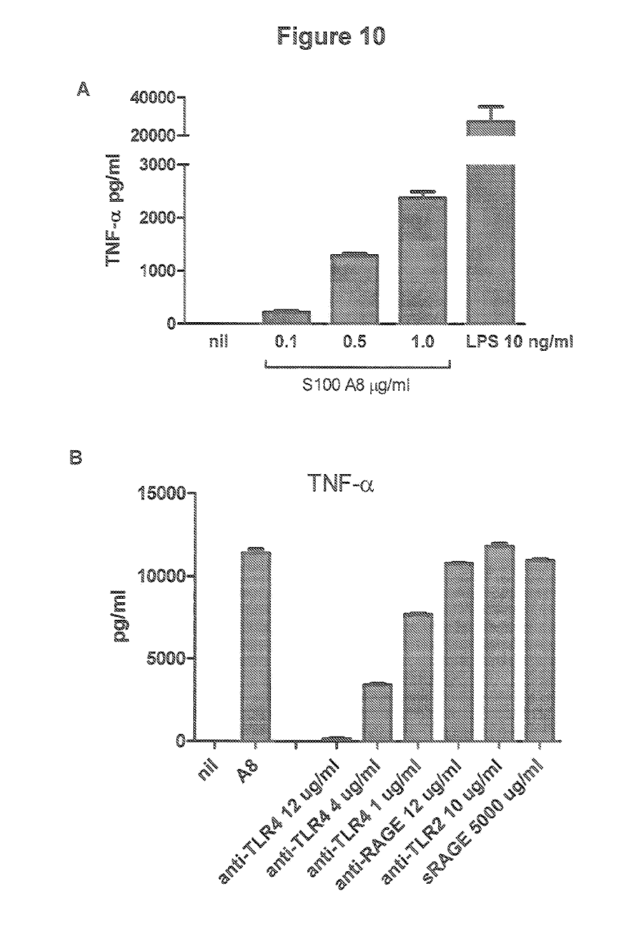

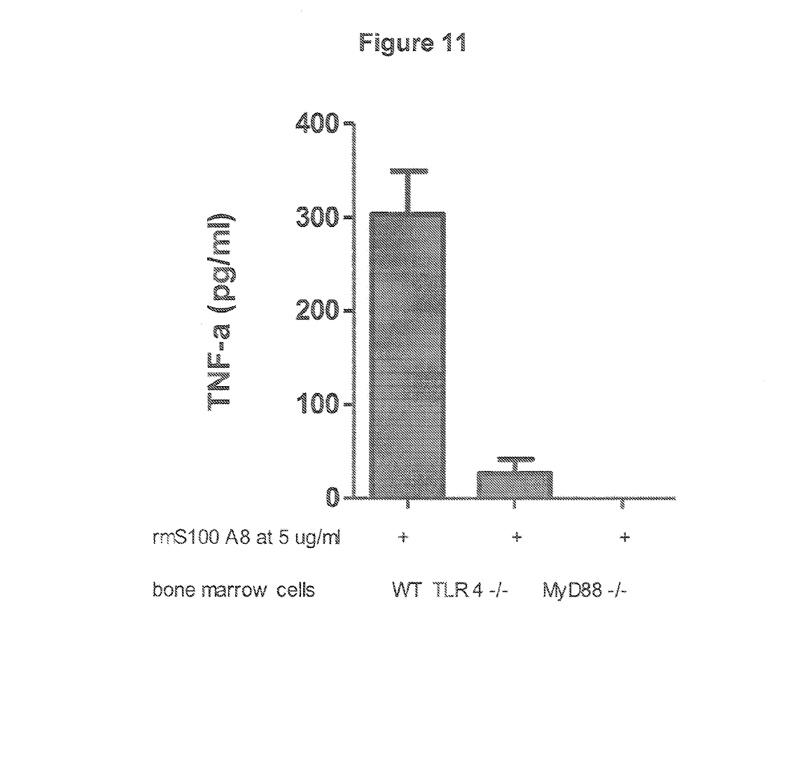

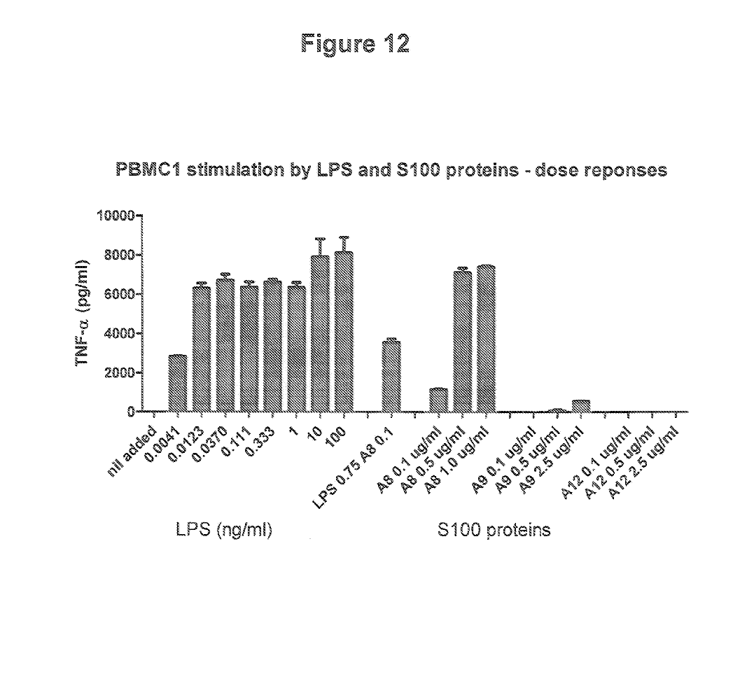

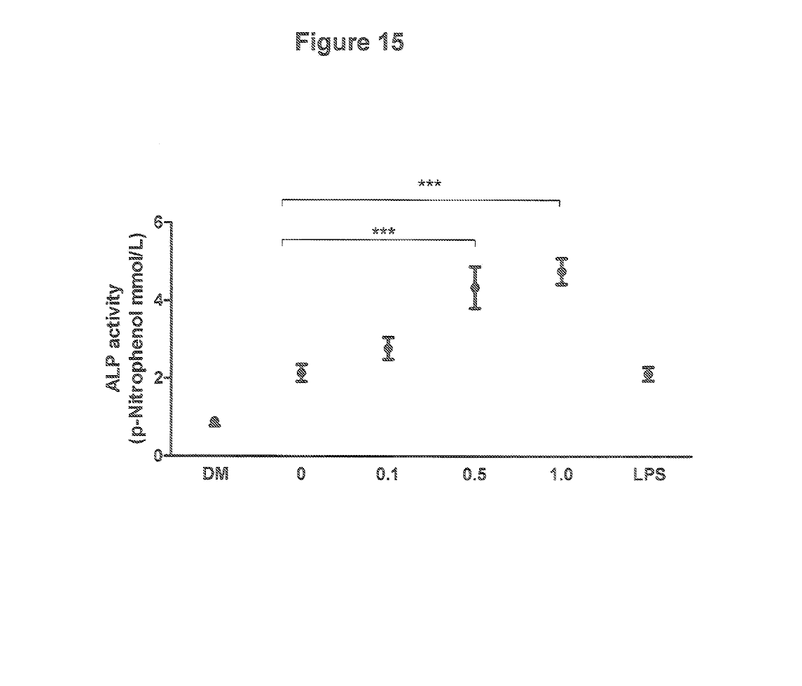

Tissue injury leads to the release of damage-associated molecular patterns (DAMPs), which have been shown to act on neutrophils, monocytes and macrophages, and many other cell types. Our observation that only traumatically fractured bone supernatants promoted osteogenesis (PCT/GB2010/001340; Glass et al 2011, PNAS 108(4): 1585-90) led us to postulate that this activity may be related to the release of alarmins (including HMGB1, HMGN1, S100A8, S100A9, S100A8/9 and S100A12), a subgroup of DAMPs. Measurement of S100 A8/9 levels by ELISA in the human fracture supernatants showed that S100 A8&9 levels correlated well with the osteogenic activity (FIG. 8). We also found increased expression of S100A8 at the fracture site compared to soft tissue dissection alone at 12 hr post injury (FIG. 9A) and two peaks in circulating S100A8/9 levels in the serum at 15 min and 24 hr post injury, again higher in the fracture animals (FIG. 9B). The combined data from human specimens and our murine model confirm the importance of alarmins, as exemplified by S100A8, in fractures. The addition of rhS100A8 to human peripheral blood mononuclear cells (PBMCs) resulted in production of TNF-α (FIG. 10A). S100A9 had a much lower effect than S100A8 (FIG. 12) and S100A8/9 had no activity, as previously reported (Vogl et al. Nature Medicine 13: 1042-49). The supernatant from the PBMC stimulated with S100A8 in turn led to osteogenesis (FIG. 15), which was abrogated by the addition of anti-TNF-α.

S100A8 and A9 are constitutively expressed in bone and cartilage cells. S100A8 but not A9 is seen in pre-osteogenic cells, whereas osteoclasts have high expression of both. Only S100A8 is detected in human bone marrow stromal cells, with high levels in preosteogenic stromal cells, which decline in more committed pre-osteoblastic cells, thus suggesting a role for S100A8 in osteoblast differentiation (Perera et al, Immunology and Cell Biology 2010 88: 41-49). In mice, S100A8 but not A9 is expressed in hypertrophic chondrocytes in the growth plate but not in the proliferative zone. Thus S100A8 is associated with osteoblast differentiation and both S100A8 and A9 contribute to calcification of the cartilage matrix and its replacement with trabecular bone and to regulation of bone resorption (Zreiqat et al, 2007 J Mol Histol 38: 381-91). S100A12 activates mast cells to release pro-inflammatory mediators. Monocytes and neutrophils are the primary source of calgranulins, which are secreted following cellular activation or released following cell necrosis. S100A8 is a potent anti-oxidant and can limit tissue damage from reactive oxygen species. Thus calgranulins contribute to the modulation and restoration of normal homeostasis in the resolution phase of inflammation but their excessive production may contribute to some facets of chronic inflammation. We found that the levels of S100A8&9 in the supernatants from human tibial fracture fragments correlated with their osteogenic activity. Furthermore, we found increased expression of S100A8 in the perifracture soft tissues over time in our murine model but a much lower increase in mice which underwent identical soft tissue dissection, including periosteal stripping, but the tibia was not fractured.

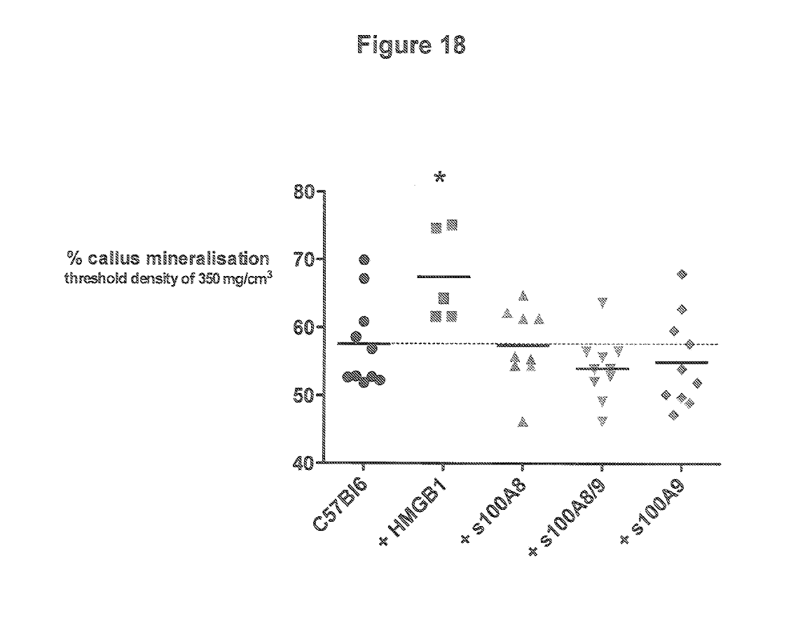

Contrary to previous reports (Andersson U, et al. High mobility group 1 protein (HMG-1) stimulates proinflammatory cytokine synthesis in human monocytes. J Exp Med 2000; 192:565), highly purified HMGB-1 did not lead to TNF-α production by PBMC. This can be explained by the fact that in vivo HMGB1 acts in combination with multiple other molecules (Bianchi J Leukoc Biol 2009 86: 573-6 Sims et al Annu Rev Immunol 2010). Furthermore, we did not find a correlation between the osteogenic activity of the fractured bone supernatants and their levels of HMGB1. However, exogenous addition of HMGB1 at the fracture site at 1 mg/ml at days 0 and 1 resulted in significantly (p=0.02) accelerated healing when assessed 28 days post fracture (FIG. 18).

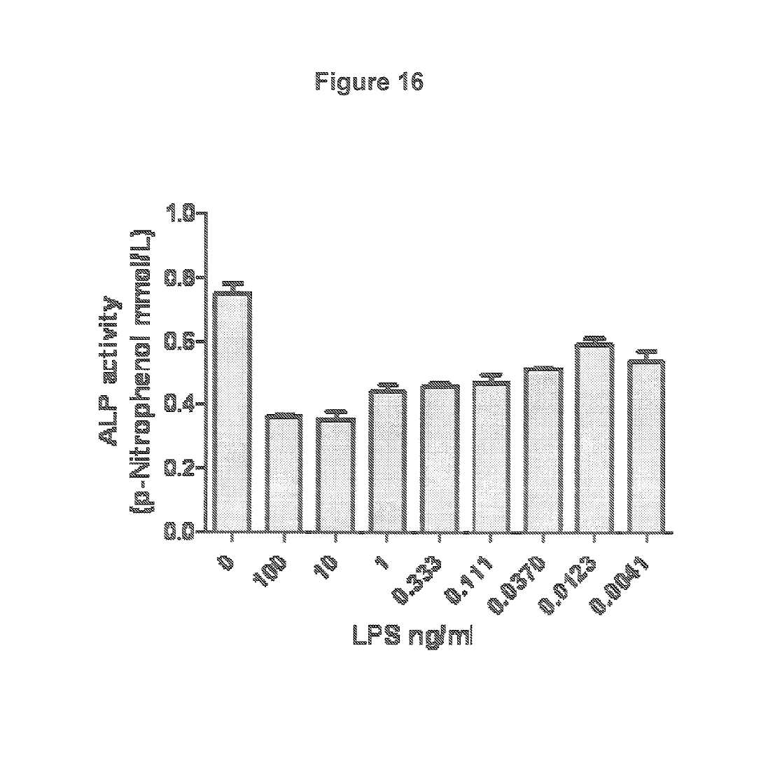

The osteogenic activity of the alarmins we have studied appears to be specific as LPS, which also leads to pro-inflammatory cytokine release, did not lead to this effect (FIG. 15 and FIG. 16).

TLR-2, TLR-4 and RAGE Ligands

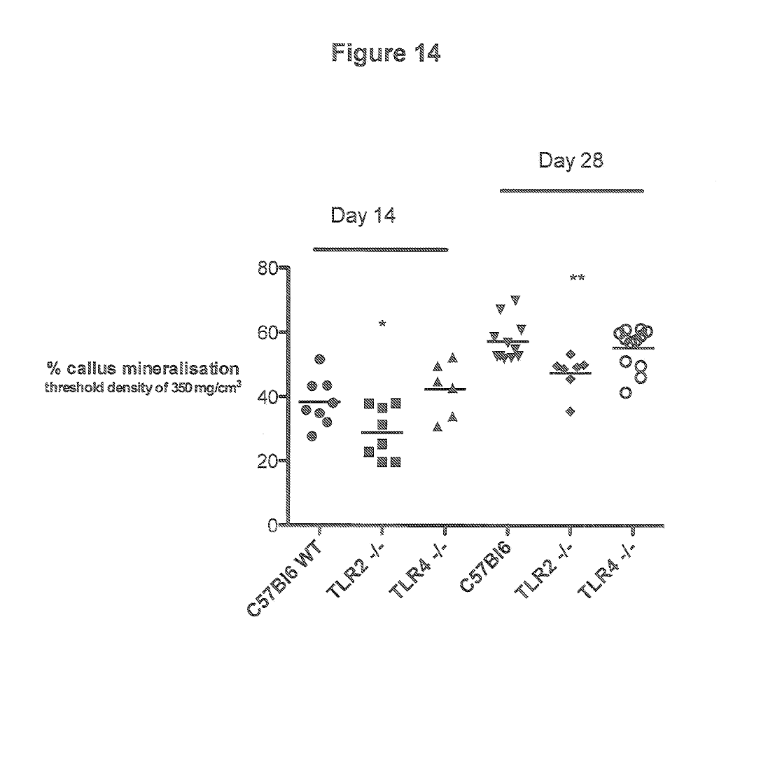

Key receptors on cells include the Toll-like receptors, which are important receptors for the alarmins, especially TLR-2 and TLR-4. We examined the healing of mice deficient in TLR-4 or TLR-2. Whilst osteogenesis in TLR-4 deficient animals appeared to proceed normally, healing at day 28 was significantly impaired in TLR-2 deficient mice (FIG. 14). It is not clear at present what aspect of endochondral bone healing is crucially dependent on TLR-2. However, it is possible that exogenous addition of TLR-2 ligands may enhance healing. There are a variety of TLR-2 ligands as in vivo TLR-2 heterodimerises with TLR-1 or TLR-6. Known TLR-2 ligands include heat killed bacteria, mycobacterial lipoglycans, P. gingivalis, LPS, lipoteichoic acids, peptidogycans, zymosan and the synthetic lipoproteins such as Pam3CSK4. We have shown that the major receptor for S100A8 is TLR-4 (FIG. 10B). Whilst TLR-4 knockout mice did not exhibit impaired healing, it is possible that TLR-4 ligands may enhance aspects of fracture healing. Similarly, a major receptor for HMGB1 is RAGE. When we added exogenous HMGB1, there was accelerated healing in our murine model.

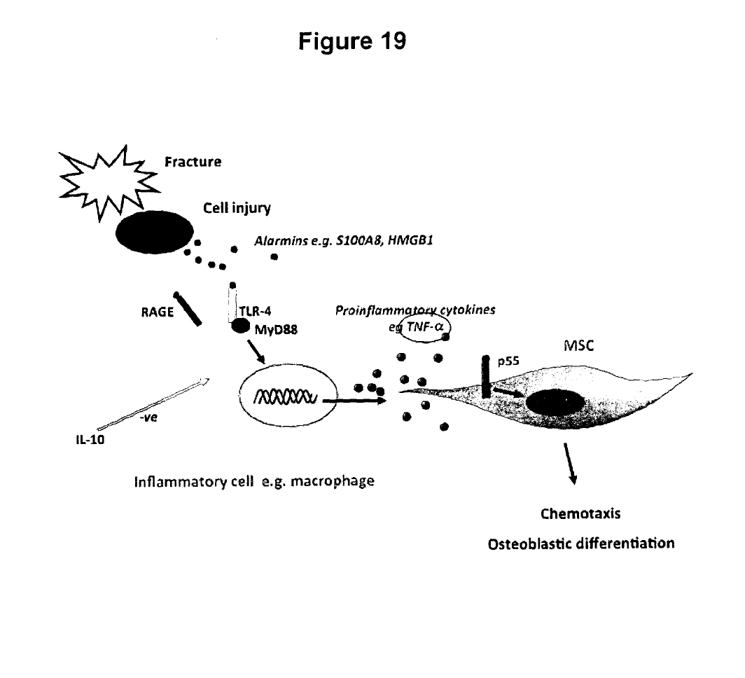

We have defined the early fracture healing pathway (FIG. 19) and hence identified a series of therapeutic targets for accelerating fracture healing, bone formation and remodeling. Upregulation of inflammation accelerates fracture healing and TNF-α is a key cytokine.

### REFERENCES

1. Melton, L. J., 3rd, Crowson, C. S. & O'Fallon, W. M. Fracture incidence in Olmsted County, Minn.: comparison of urban with rural rates and changes in urban rates over time. Osteoporos Int 9, 29-37 (1999).
2. Harvey, N., Dennison, E. & Cooper, C. Osteoporosis: impact on health and economics. Nat Rev Rheumatol 6, 99-105.
3. Hashimoto, J., et al. Inhibitory effects of tumor necrosis factor alpha on fracture healing in rats. Bone 10, 453-457 (1989).
4. Yoshikawa, H., Hashimoto, J., Masuhara, K., Takaoka, K. & Ono, K. Inhibition by tumor necrosis factor of induction of ectopic bone formation by osteosarcoma-derived bone-inducing substance. Bone 9, 391-396 (1988).
5. Moore, K. W., O'Garra, A., de Waal Malefyt, R., Vieira, P. & Mosmann, T. R. Interleukin-10. Annu Rev Immunol 11, 165-190 (1993).
6. de Waal Malefyt, R., Abrams, J., Bennett, B., Figdor, C. G. & de Vries, J. E. Interleukin 10(IL-10) inhibits cytokine synthesis by human monocytes: an autoregulatory role of IL-10 produced by monocytes. J Exp Med 174, 1209-1220 (1991).
7. Fortunato, S. J., Menon, R., Swan, K. F. & Lombardi, S. J. Interleukin-10 inhibition of interleukin-6 in human amniochorionic membrane: transcriptional regulation. Am J Obstet Gynecol 175, 1057-1065 (1996).
8. Fiorentino, D. F., Zlotnik, A., Mosmann, T. R., Howard, M. & O'Garra, A. IL-10 inhibits cytokine production by activated macrophages. J Immunol 147, 3815-3822 (1991).
9. DiPietro, L. A., Polverini, P. J., Rahbe, S. M. & Kovacs, E. J. Modulation of JE/MCP-1 expression in dermal wound repair. Am J Pathol 146, 868-875 (1995).
10. Sato, Y., Ohshima, T. & Kondo, T. Regulatory role of endogenous interleukin-10 in cutaneous inflammatory response of murine wound healing. Biochem Biophys Res Commun 265, 194-199 (1999).
11. Dimitriou, R., Tsiridis, E. & Giannoudis, P. V. Current concepts of molecular aspects of bone healing. Injury 36, 1392-1404 (2005).
12. Mountziaris, P. M. & Mikos, A.G. Modulation of the inflammatory response for enhanced bone tissue regeneration. Tissue Eng Part B Rev 14, 179-186 (2008).
13. Cho, T. J., Gerstenfeld, L. C. & Einhorn, T. A. Differential temporal expression of members of the transforming growth factor beta superfamily during murine fracture healing. J Bone Miner Res 17, 513-520 (2002).
14. Kon, T., et al. Expression of osteoprotegerin, receptor activator of NF-kappaB ligand (osteoprotegerin ligand) and related proinflammatory cytokines during fracture healing. J Bone Miner Res 16, 1004-1014 (2001).
15. Lehmann, W., et al. Tumor necrosis factor alpha (TNF-alpha) coordinately regulates the expression of specific matrix metalloproteinases (MMPS) and angiogenic factors during fracture healing. Bone 36, 300-310 (2005).
16. Gerstenfeld, L. C., at al. Impaired fracture healing in the absence of TNF-alpha signaling: the role of TNF-alpha in endochondral cartilage resorption. J Bone Miner Res 18, 1584-1592 (2003).
17. Rifas, L. T-cell cytokine induction of BMP-2 regulates human mesenchymal stromal cell differentiation and mineralization. J Cell Biochem 98, 706-714 (2006).
18. Takamiya, M., Fujita, S., Saigusa, K. & Aoki, Y. A study on mRNA expressions of interleukin 10 during fracture healing for wound age determination. Leg Med (Tokyo) 10, 131-137 (2008).
19. Riley, J. K., Takeda, K., Akira, S. & Schreiber, R. D. Interleukin-10 receptor signaling through the JAK-STAT pathway. Requirement for two distinct receptor-derived signals for anti-inflammatory action. J Biol Chem 274, 16513-16521 (1999).
20. Yamamoto, T., Eckes, B. & Krieg, T. Effect of interleukin-10 on the gene expression of type I collagen, fibronectin, and decorin in human skin fibroblasts: differential regulation by transforming growth factor-beta and monocyte chemoattractant protein-1. Biochem Biophys Res Commun 281, 200-205 (2001).
21. Moroguchi, A., et al. Interleukin-10 suppresses proliferation and remodeling of extracellular matrix of cultured human skin fibroblasts. Eur Surg Res 36, 39-44 (2004).
22. Eming, S. A., et al. Accelerated wound closure in mice deficient for interleukin-10. Am J Pathol 170, 188-202 (2007).
23. Gordon, A., et al. Permissive environment in postnatal wounds induced by adenoviral-mediated overexpression of the anti-inflammatory cytokine interleukin-10 prevents scar formation. Wound Repair Regen 16, 70-79 (2008).
24. Peranteau, W. H., et al. IL-10 overexpression decreases inflammatory mediators and promotes regenerative healing in an adult model of scar formation. J Invest Dermatol 128, 1852-1860 (2008).
25. Liechty, K. W., Kim, H. B., Adzick, N. S. & Crombleholme, T. M. Fetal wound repair results in scar formation in interleukin-10-deficient mice in a syngeneic murine model of scarless fetal wound repair. J Pediatr Surg 35, 866-872; discussion 872-863 (2000).
26. Ho, A. S., et al. A receptor for interleukin 10 is related to interferon receptors. Proc Natl Acad Sci USA 90, 11267-11271 (1993).
27. Samarasinghe, R., et al. Induction of an anti-inflammatory cytokine, IL-10, in dendritic cells after toll-like receptor signaling. J Interferon Cytokine Res 26, 893-900 (2006).
28. Harry, L. E., et al. Comparison of the healing of open tibial fractures covered with either muscle or fasciocutaneous tissue in a murine model. J Orthop Res 26, 1238-1244 (2008).

### EXAMPLE 2

### In Vivo Local Administration of TNF-α at Fracture Site

Our in vitro data (see PCT/GB2010/001340 and Glass G, Chan J, Freidin A, Feldmann M, Horwood N, Nanchahal J. TNF-α promotes fracture repair by augmenting the recruitment and differentiation of muscle-derived stromal cells. Proceedings of the National Academy of Sciences, USA. 2011. 108(4): 1585-1590) show that TNF-α may potentially be used in vivo to accelerate fracture healing. The optimal dose range in the in vitro data was fairly narrow, with the optimal in vitro dose being 0.1-1 ng/ml and a 10 fold increase beyond this range (10 ng/ml) resulting in a significant diminution in effect. We also showed that at 50 ng/ml (1 ng administered to the fracture site in 20 microlitres of phosphate buffered saline) at the tibial fracture site in a mouse (of average weight 20 g) resulted in significantly accelerated healing of the fracture.

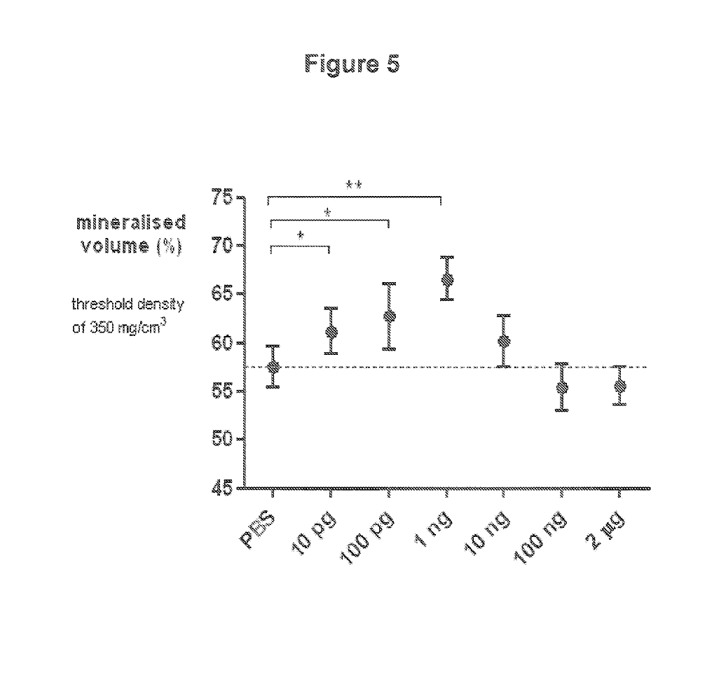

The data provided in FIG. 5 shows that in vivo the dose range is actually quite wide, with doses of 10 pg to 1 ng administered at the fracture site in our murine model resulting in significantly accelerated healing compared to control animals where only the carrier (phosphate buffered saline) was administered at the fracture site.

Extrapolating to the human by body weight (average man 70 kg) the average man is 3500 fold larger than the mouse. Extrapolating to the human by cross sectional area of the tibial shaft, the average human is 900 fold larger. Therefore, we would predict that a dose of the order of 0.01-3.5 micrograms delivered at the fracture site in the human is likely to significantly accelerate fracture healing.

Previous clinical trials have demonstrated that single doses of TNF-alpha up to 400 micrograms administered subcutaneously or intramuscularly in patients with disseminated cancer are safe (Saks, S. and M. Rosenblum, Recombinant human TNF-alpha: preclinical studies and results from early clinical trials. Immunol Ser, 1992. 56: p. 567-87). The highest doses administered repeatedly resulted in clinically insignificant thrombocytopaenia when given intramuscularly and minor local reactions when administered subcutaneously (Blick, M., et al., Phase I study of recombinant tumor necrosis factor in cancer patients. Cancer Res, 1987. 47(11): p. 2986-9). Therefore, we would predict that the dose range we would use therapeutically projected from our murine data when administered locally at the fracture site is unlikely to result in significant adverse effects in humans.

The greatest clinical need for accelerating fracture healing is in patients with osteoporosis. Fragility fractures in osteoporotic bone heal slowly and surgical fixation is difficult. Worldwide 100-200 million people are at risk of fragility fractures and the cost in 2000 was £1.7 billion in the UK alone (Harvey, N., E. Dennison, and C. Cooper, Osteoporosis: impact on health and economics. Nat Rev Rheumatol. 2010; 6(2): p. 99-105). By 2025, the annual global incidence of hip fractures, which are the most severe with a mortality rate of 24% in the first year, is estimated at 4.5-6.3 million. The lifetime risk of clinically significant fragility fractures is 40%, equivalent to that for cardiovascular disease. There is currently no technique for accelerating healing of fragility fractures. The only approved biological therapy for accelerating fracture healing is the exogenous addition of bone morphogenetic proteins (BMPs) for tibial fractures. However clinical results are less impressive than in preclinical animal models (Garrison, K. R., et al., Bone morphogenetic protein (BMP) for fracture healing in adults. Cochrane Database Syst Rev. 6: p. CD006950). Alternative techniques of expanding autologous mesenchymal stromal cells, including those transduced to over express BMPs, face significant translational hurdles and cost implications. Moreover, osteoporosis is associated with reduced bone healing capacity and a weakened bone structure (Barrios, C., et al., Healing complications after internal fixation of trochanteric hip fractures: the prognostic value of osteoporosis. J Orthop Trauma, 1993. 7(5): p. 438-42). This results in a dramatically increased failure rate of up to 50% following surgical fixation due to a ‘pull out’ or ‘cut-through’ phenomenon whereby the screws used to secure the supporting plate fail to gain sufficient purchase in the osteoporotic bone (Cornell, C. N., Internal fracture fixation in patients with osteoporosis. J Am Acad Orthop Surg, 2003. 11(2): p. 109-19 Kim, W. Y., et al., Failure of intertrochanteric fracture fixation with a dynamic hip screw in relation to pre-operative fracture stability and osteoporosis. Int Orthop, 2001. 25(6): p. 360-2). Rates of recovery and mobilisation are limited by the time required for fracture healing as premature loading leads to implant failure; this failure accounts for the excessive morbidity and mortality seen in this vulnerable group of patients. Therefore, there is an urgent unmet need to develop strategies to accelerate healing of fragility fractures.

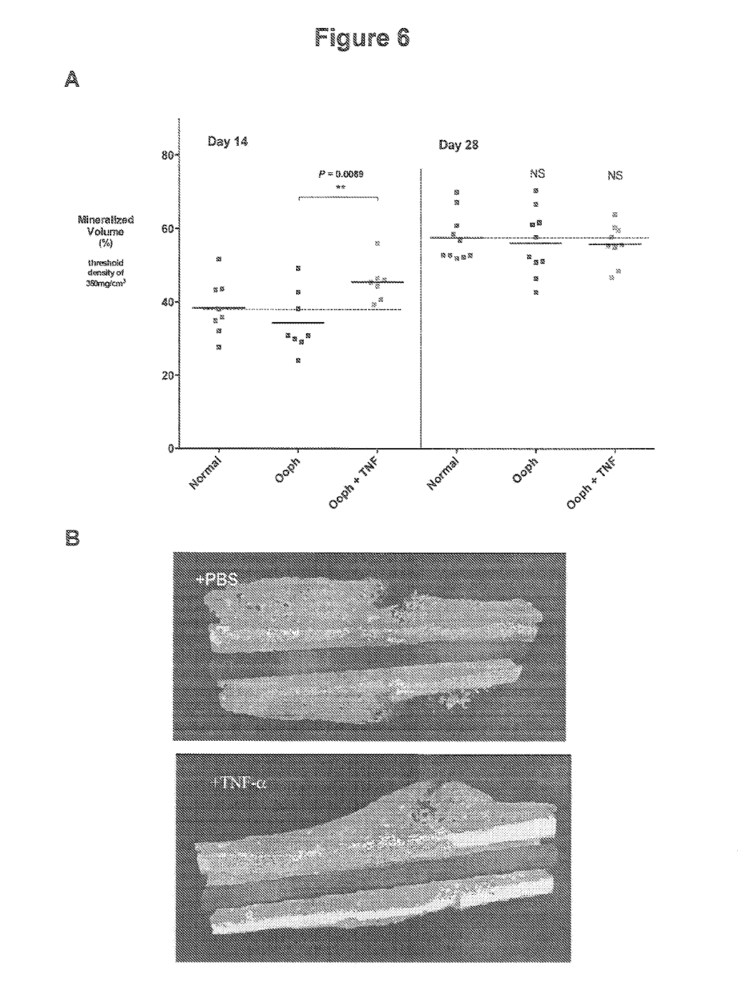

We have investigated the effect of adding recombinant human TNF-α at the fracture site in mice rendered osteoporotic by oophorectomy 4 weeks previously. Oophrectomy led to a small reduction in the rate of healing compared to wild type control mice and we found significantly accelerated fracture healing at 2 weeks post fracture in osteoporotic mice compared to the administration of phosphate buffered saline alone (FIG. 6).

### EXAMPLE 3

### Damage-Associated Molecular Patterns (DAMPs), or Alarmins, in Fracture Repair

DAMPS, or ‘alarmins’, are released upon injury, eliciting innate and adaptive immune responses [1, 2]. The best characterised are HMGB1 and S100 proteins, both of which are highly pro-inflammatory in the extracellular space [3-8].

Alarmins, including HMGB1, have been described to play a pathogenic role in sterile and infection induced inflammation [11-13]. To date, studies have focussed on the modulation or attenuation of alarmins, including HMGB1 and S100 proteins, as a potential therapeutic strategy to control infection and injury-elicited inflammation, as well as inflammatory diseases [9, 10].

However, as alarmins exhibit pro-inflammatory properties, they are likely linked to the initiation of reparative processes. Recently, HMGB1 has been shown to improve the repair of the myocardium post infarction in mice by promoting the recruitment of resident cardiac stem cells as well as their proliferation and differentiation [20]. HMGB1 may have a similar reparative role in fracture repair. However, to date, no study has investigated the role of alarmins in this context and the limited data on the role of alarmins in bone biology are conflicting.

HMGB1 has recently been shown to act as a bone active cytokine [16]. It is expressed and released by osteoblasts and osteoclasts and both express RAGE [17]. While HMGB1 has been shown to stimulate osteoblastic differentiation [18], activation of RAGE, one of the receptors of HMGB1 [14], stimulates osteoclastogenesis and impairs matrix mineralisation [15]. HMGB1 has also been shown to inhibit the proliferation of mesenchymal stromal cells and promote migration and differentiation along the osteoblastic pathway [21]. A study of HMGB1 knockout mice suggested that HMGB1 secreted by chondrocytes may regulate endochondral ossification and function as a chemoattractant for osteoblasts and osteoclasts as well as endothelial cells during embryonic osteogenesis [22], but to date there are no publications as to its role in postnatal fracture repair.

S100A8 and A9 have been reported to be highly expressed in osteoblasts and contribute to mineralisation of cartilage matrix and osteoblastic differentiation [19].

We have previously shown that only supernatants from fractured bone fragments but not surgically cut human bone fragments promote migration of stromal cells and then their osteogenic differentiation. We also demonstrated that these effects were in the main due to the presence of pro-inflammatory cytokines, in particular TNF-α, in the fracture supernatants [23].

We provide evidence herein that fracture of the bone leads to the release of alarmins including S100A8, which in turn lead to the production of TNF-α by inflammatory cells. We also present evidence that exogenous addition of HMGB1 accelerates fracture healing and hence they may be a potential therapeutic.

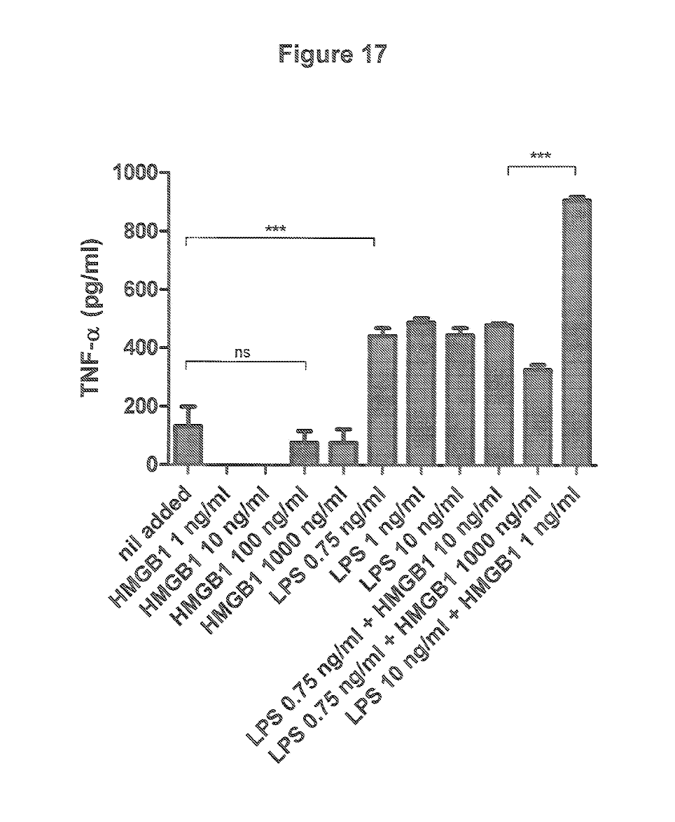

FIG. 8 shows that S100A8/9 levels correlate with osteogenic activity of human fracture supernatants. FIG. 9A shows that S100 A8 expression in the mouse fracture (tibial osteotomy) model is induced especially by fracture. FIG. 9B shows increased circulating levels of S100A8&9 at 15 min and 24 hr post injury, higher in the fracture group than in those animals only undergoing soft tissue dissection. HMGB1 is not pro-inflammatory in isolation in vitro and requires complex formation with other molecules [24-26]. FIG. 17 illustrates that the addition of LPS induces a synergistic effect with HMGB1 in TNF-α production by peripheral blood mononuclear cells (PBMCs). FIGS. 10A and B show that S100 A8 stimulates monocytes to produce TNF-α in a dose dependent manner via TLR-4, not TLR2 or RAGE.

We have shown that S100 A9 and S100 A12 are much less potent than S100 A8 for PBMC stimulation in vitro. FIG. 15 shows that supernatant from PBMCs stimulated with S100A8 is osteogenic. FIG. 15 also shows that the effect is not only TNFα-dependent as LPS-stimulated monocyte supernatant is not osteogenic. This suggests that the osteogenic activity is specific to certain alarmins such as S100A8. The lack of osteogenic response of human muscle-derived stromal cells exposed to supernatants from PBMC stimulated with LPS was consistent over a wide dose range of LPS (FIG. 16).

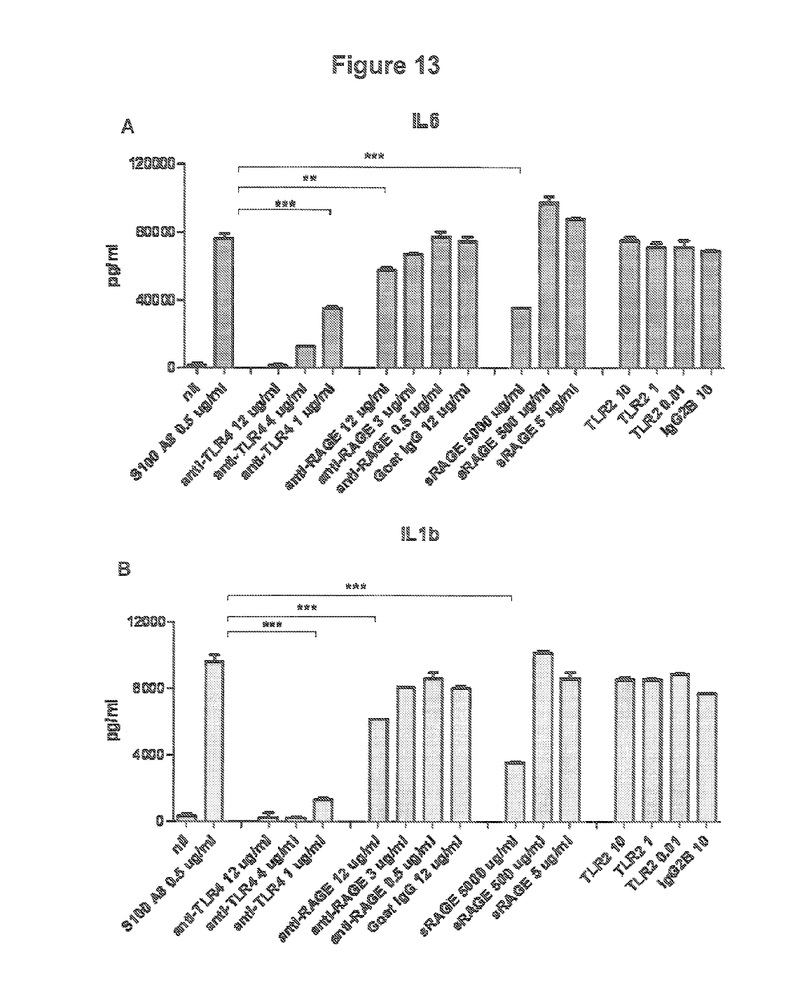

The other pro-inflammatory cytokines that we had previously found to be osteogenic in vitro were IL-6 and IL-1β, and these are also produced by monocytes via TLR4 and also RAGE, but not TLR2 (FIG. 13).

FIG. 18 shows that addition of exogenous alarmin rmHMGB1 locally at the fracture site in the murine model of endochondral fracture healing improves callus mineralisation in viva

### REFERENCES

1. Oppenheim, J. J., et al., Alarmins initiate host defense. Adv Exp Med Biol, 2007. 601: p. 185-94.
2. Bianchi, M. E., DAMPs, PAMPs and alarmins: all we need to know about danger. J Leukoc Biol, 2007. 81(1): p. 1-5.
3. Wang, H., et al., HMG-1 as a late mediator of endotoxin lethality in mice. Science, 1999. 285(5425): p. 248-51.
4. Andersson, U., et al., High mobility group 1 protein (HMG-1) stimulates proinflammatory cytokine synthesis in human monocytes. J Exp Med, 2000. 192(4): p. 565-70.
5. Bianchi, M. E. and A. A. Manfredi, Immunology. Dangers in and out. Science, 2009. 323(5922): p. 1683-4.
6. Roth, J., et al., Phagocyte-specific S100 proteins: a novel group of proinflammatory molecules. Trends Immunol, 2003. 24(4): p. 155-8.
7. Foell, D., et al., S100 proteins expressed in phagocytes: a novel group of damage-associated molecular pattern molecules. J Leukoc Biol, 2007. 81(1): p. 28-37.
8. Levy, R. M., et al., Systemic inflammation and remote organ injury following trauma require HMGB1. Am J Physiol Regul Integr Comp Physiol, 2007. 293(4): p. R1538-44.
9. Piccinini, A. M. and K. S. Midwood, DAMPening inflammation by modulating TLR signalling. Mediators Inflamnn, 2010. 2010.
10. Zhu, S., et al., High mobility group box 1 protein as a potential drug target for infection- and injury-elicited inflammation. Inflamm Allergy Drug Targets, 2010. 9(1): p. 60-72.
11. Yang, H. and K. J. Tracey, Targeting HMGB1 in inflammation. Biochim Biophys Acta, 2010. 1799(1-2): p. 149-56.
12. Andersson, U. and H. E. Harris, The role of HMGB1 in the pathogenesis of rheumatic disease. Biochim Biophys Acta, 2010. 1799(1-2): p. 141-8.
13. Wang, H., et al., HMGB1 as a potential therapeutic target. Novartis Found Symp, 2007. 280: p. 73-85; discussion 85-91, 160-4.
14. Kokkola, R., et al., RAGE is the major receptor for the proinflammatory activity of HMGB1 in rodent macrophages. Scand J Immunol, 2005. 61(1): p. 1-9.
15. Franke, S., et al., Advanced glycation endproducts influence the mRNA expression of RAGE, RANKL and various osteoblastic genes in human osteoblasts. Arch Physiol Biochem, 2007. 113(3): p. 154-61.
16. Yang, J., et al., HMGB1 is a bone-active cytokine. J Cell Physiol, 2008. 214(3): p. 730-9.
17. Charoonpatrapong, K., et al., HMGB1 expression and release by bone cells. J Cell Physiol, 2006. 207(2): p. 480-90.
18. Bidwell, J. P., J. Yang, and A. G. Robling, Is HMGB1 an osteocyte alarmin? J Cell Biochem, 2008. 103(6): p. 1671-80.
19. Zreiqat, H., et al., S100A8/S100A9 and their association with cartilage and bone. J Mol Histol, 2007. 38(5): p. 381-91.
20. Germani, A., F. Limana, and M. C. Capogrossi, Pivotal advances: high-mobility group box 1 protein—a cytokine with a role in cardiac repair. J Leukoc Biol, 2007. 81(1): p. 41-5.
21. Meng, E., et al., High mobility group box 1 protein inhibits the proliferation of human mesenchymal stem cells and promotes their migration and differentiation along osteoblastic pathway. Stem Cells Dev, 2008. 17(4): p. 805-13.
22. Taniguchi, N., et al., Stage-specific secretion of HMGB1 in cartilage regulates endochondral ossification. Mol Cell Biol, 2007. 27(16): p. 5650-63.
23. Glass, G. E., et al., TNF-alpha promotes fracture repair by augmenting the recruitment and differentiation of muscle-derived stromal cells. Proc Natl Acad Sci USA, 2011. 108(4): p. 1585-90.
24. Bianchi, M. E., HMGB1 loves company. J Leukoc Biol, 2009. 86(3): p. 573-6.
25. Hreggvidsdottir, H. S., et al., The alarmin HMGB1 acts in synergy with endogenous and exogenous danger signals to promote inflammation. J Leukoc Biol, 2009. 86(3): p. 655-62.
26. Yanai, H., et al., HMGB proteins function as universal sentinels for nucleic-acid-mediated innate immune responses. Nature, 2009. 462(7269): p. 99-103.

1. A method of promoting bone formation in a patient at a site in need thereof, the method comprising the step of locally administering a pro-inflammatory compound to the site, wherein the pro-inflammatory compound is selected from one or more of TNF-α at optimal osteogenic dose of 0.5 to 50 ng/kg of patient body weight, or 0.01 to 3.5 μg, or 1 ng/ml or similar; IL-1β at optimal osteogenic dose of 0.1 ng/ml or similar; alarmins eg HMGB1, HMGN1, S100A8, S100A9, S100A8/9, S100A12, heat shock proteins, lactoferrin, cathelicidins, a-defensins, matrix components including versican, biglycan, fragments of hyaluronic acid and heparan sulphate; and TLR-2 and/or TLR-4 ligands.

2-3. (canceled)

4. The method of claim 1, further comprising for administering a combination of said pro-inflammatory compounds to the patient, for example an administration of TNF-α or IL-1β followed by administration of an alarmin, for example HMGB1 or S100A8, to upregulate the local effect.

5. The method of claim 1, wherein the site is a site of injury, a site of surgical intervention, a site requiring bone fusion or comprising damaged bone, eroded bone or bone defects.

6. The method of claim 5, wherein the injury is a fracture of a bone.

7. The method of claim 5, wherein the surgical intervention is an osteotomy, a bone graft, an excision of bone from a donor site for a bone graft, the insertion of an implant into, around and/or adjacent to a bone or the fixing of an implant to a bone.

8. The method of claim 1, wherein the promotion of bone formation aids in repairing bone, accelerating bone formation, increasing cortical bone volume, increasing cortical bone mineral content, increasing bone mineral density at the site, increasing mineralised volume of the healing bone, the mineralised bone volume fraction and/or tissue mineral density, accelerating remodeling of the callus at the site, for example a fracture site, and/or accelerating remodelling of any newly formed bone at the site.

9. The method claim 7, wherein the implant is selected from, the group comprising a joint replacement, a dental implant, a pin, a plate, a screw, an intramedullary device and/or an intraosseous device.

10. The method of claim 7, wherein adherence of the implant to the bone is strengthened in comparison with adherence of an implant to bone in the absence of the method of claim 7.

11. The method of claim 7, wherein the implant has a reduced tendency to loosening from the site of insertion in comparison with an implant inserted in the absence of the method of claim 7.

12. The method claim 6, wherein the fractured bone has a disrupted or damaged periosteum and/or endosteum.

13. The method of claim 6, wherein the fractured bone has an intact periosteum and/or endosteum.

14. The method of claim 1, wherein the patient has compromised bone due to metabolic bone disorders hereditary bone conditions, osteoporosis, infection, malignant or benign tumours affecting bone, bone affected by chemotherapy, radiotherapy and/or disuse.

15. (canceled)

16. The method of claim 14, wherein the newly formed bone has improved bone quality, quantity, density and shorter healing times in comparison with the compromised bone previously present at the site.

17. The method of claim 1, wherein the promotion of bone formation augments and/or accelerates bone formation during distraction lengthening.

18. The method of claim 1, wherein the promotion of bone formation accelerates bone formation in tissue engineered constructs.

19. The method of claim 1, wherein the compound is administered to the site, in the form of a liquid for injection or otherwise, an infusion, a cream, a lozenge, a gel, a lotion, a paste or a liquid.

20. The method of claim 1, wherein the compound is administered to the site in a controlled release preparation which is biocompatible, and which is liquid at low temperature but assumes gel characteristics at body temperature.

21. The method of claim 3, wherein the pro-inflammatory compound is administered immediately following injury or surgery.

22. The method of claim 3, wherein the pro-inflammatory compound is administered between one hour and one year after the injury or surgical intervention.

23. The method of claim 21, wherein the pro-inflammatory compound is administered at the time of surgical intervention or injury.

24. A kit of parts comprising a surgical implant in combination with a pro-inflammatory compound as defined in claim 1.

25. The kit of claim 24 further comprising cement suitable for bonding the surgical implant to bone.

26. The kit of claim 25, wherein the pro-inflammatory compound is dispersed within the cement.

27. The kit of claim 24, wherein the surgical implant is coated with the pro-inflammatory compound.

28. The kit of claim 27, wherein the pro-inflammatory compound is covalently bound to the surgical implant.

29. The kit of claim 24, wherein the surgical implant is selected from the group comprising a joint replacement, a plate, a pin, a screw, a dental implant, an intramedullary device or an intraosseous device.

30. The method of claim 1, wherein the patient is selected from the group comprising mammals, birds, amphibians, fish and reptiles.

31. The method of claim 30, wherein the mammal is selected from the group comprising humans, apes, monkeys, sheep, cattle, goats, swine, horses, dogs, cats, mice, rats, guinea pigs, hamsters, rabbits and gerbils.

32-34. (canceled)

35. The kit of claim 26, wherein the surgical implant and/or cement is coated with the pro-inflammatory compound.

36. The kit of claim 35, wherein the pro-inflammatory compound is covalently bonded to the surgical implant and/or cement.

37. The kit of claim 24 wherein the patient is selected from the group comprising mammals, birds, amphibians, fish and reptiles.

38. The kit of claim 25, wherein the mammal is selected from the group comprising humans, apes, monkeys, sheep, cattle, goats, swine, horses, dogs, cats, mice, rats, guinea pigs, hamsters, rabbits and gerbils.
